(19) United States
(12) Patent Application Publication
(10) Pub. No.: US 2014/0274768 A1
(43) Pub. Date: Sep. 18, 2014

(54) Glycoforms of MUC5AC and Endorepellin and Biomarkers for Mucinous Pancreatic Cysts

(71) Applicant: Van Andel Research Institute, Grand Rapids, MI (US)

(72) Inventor: Brian Brummeler Haab, Jenison, MI (US)

(21) Appl. No.: 14/203,049

(22) Filed: Mar. 10, 2014

Related U.S. Application Data
(60) Provisional application No. 61/783,361, filed on Mar. 14, 2013.

(51) Int. Cl. G01N 33/574

(52) U.S. Cl. CPC G01N 33/57438
USPC 506/9; 435/7.92

(57) ABSTRACT

The present invention relates to a method for diagnosing a subject with a malignant pancreatic cyst, the method comprising, obtaining a pancreatic cyst fluid sample from a pancreatic cyst lesion of the subject, detecting the level of MUC5AC, and endorepellin glycoforms present in the pancreatic cyst fluid sample, comparing the levels of MUC5AC and endorepellin glycoforms to a control pancreatic cyst sample level of MUC5AC and endorepellin glycoforms; and diagnosing the pancreatic cyst lesion as malignant if the levels of the MUC5AC and endorepellin glycoforms are differentially expressed compared to the levels of the MUC5AC and endorepellin glycans present in control pancreatic cyst samples.

### CROSS-REFERENCE TO RELATED APPLICATIONS

This application claims the benefit of U.S. Provisional Application Ser. No. 61/783,361, filed Mar. 14, 2013. The disclosure of this document is hereby incorporated by reference in its entirety.

### STATEMENT REGARDING FEDERALLY SPONSORED RESEARCH OR DEVELOPMENT

This invention was made with government support under grant 1U01CA152653 awarded by the National Institute of Health. The government has certain rights in the invention.

### INCORPORATION BY REFERENCE OF MATERIAL SUBMITTED ELECTRONICALLY

Incorporated by reference in its entirety is a computer-readable sequence listing submitted concurrently herewith and identified as follows: One 14 KB ASCII (Text) file named “232119-353204_ST25.txt,” created on Feb. 24, 2014, at 11:47 am.

### TECHNICAL FIELD

The present invention relates generally to biomarkers that differentiate malignant mucinous pancreatic cysts from benign pancreatic lesions and as sensitive indicators of neoplastic transformation.

### BACKGROUND

Cysts in the pancreas, which sometimes show up in the course of diagnostic imaging (1, 2), come with various levels of danger to the patient. Some might progress to invasive and lethal cancer, while others will remain indolent (3). Patients and doctors usually are not sure about which type of cyst it is and thus have trouble deciding whether it should be removed. They mainly base their decision on the size of the cyst, its location, whether it grows over time, and whether it has a solid component (4). In addition, certain molecular and cellular features of the fluid within the cyst, which can be removed by endoscopic ultrasound-guided fine-needle aspiration (EUS-FNA), may provide additional guidance. A test for carcinoembryonic antigen (CEA) distinguishes potentially malignant cysts from benign cysts with about 80% accuracy (5, 6), and the presence of abnormal cells confirms malignancy or pre-malignancy for a subset of patients (5). These tactics are useful for some patients but are not conclusive for many others (7, 8).

The cyst fluid might be the best place to look for more accurate information about pancreatic cysts. The fluid is in direct contact with the cells lining a cyst, and secretions from those cells are trapped, rather than diluted into the blood. Examples of potential biomarkers in cyst fluid include DNA mutations (9, 10), specific protein levels (11), microRNAs (12), inflammatory cytokines (13), and the presence of mucin (14, 15). A combined analysis of cytology and DNA quality showed a ˜75% accuracy for separating malignant from benign cysts (16), and the combination of CEA and kRas mutation detection gives a sensitivity of 84% and specificity of 67% (17). Broader searches by genomic, proteomic, and glycomic profiling (3, 7, 18, 19) uncovered additional candidate biomarkers to be pursued in future studies.

Prior studies addressed whether specific protein glycoforms are present predominantly in the fluid from cysts that have the potential to progress to cancer (20). The reason for investigating specific glycoforms, rather than simply the protein levels, is that pathological cells can rework the glycosylation machinery in addition to the transcription levels of proteins. For example, cancer cells and dysplastic cells can enhance N-glycan complexity through increased branching (21), produce truncated β-glycans through the lost activity of an enzyme critical for O-glycan extension (22), or increase production of Lewis structures or fucosylation (18, 23, 24). Such cancer-associated glycan alterations can affect cancer progression through changing cellular adhesiveness, migration, cytokine signaling, receptor recycling, or immune cell interactions (25). Therefore, more information about the state of a cell can be determined by detecting protein glycosylation in addition to protein abundance.

A promising means of diagnosing the type of cystic tumor is the analysis of the fluid trapped inside the cyst, which can be collected by endoscopic ultrasound fine-needle aspiration (EUS-FNA). The cytologic examination of cyst fluid has low diagnostic sensitivity, presumably because of the paucity of tumor cells within the cyst itself. Recently, molecular studies have been performed on cyst fluid samples in order to discover biomarkers secreted by the encapsulating epithelial cells that are indicative of the type of cyst. Thus far, the most accurate biomarker is carcinoembryonic antigen (CEA). A combined analysis of 12 different studies found that CEA distinguished mucin-producing (not including IPMN) from benign cysts with an average 48% sensitivity and 98% specificity (26). Other types of biomarkers that have been tested in cyst fluid include DNA quality and mutations (27), tumor-associated trypsin inhibitor (28), and the presence of mucin (29,30). Despite great initial enthusiasm for the commercially available REDPATH™ evaluation of cyst fluid, this DNA analysis appears to have significant limitations to accurately select patients who require surgery, and has not replaced CEA testing for routine diagnostic analysis (31). As set forth below, the inventors now have discovered glycosylation variants on specific proteins in cyst fluid samples that could serve as biomarkers to aid in this diagnosis.

Despite extensive research in the field of diagnostic testing for malignant transformation and expression, particularly for pancreatic cancer, there remains a need to identify novel biomarkers and methods that are capable of rapidly and accurately distinguishing between mucinous malignant pancreatic cysts and benign cysts.

### SUMMARY

The inventors have found that certain mucin and endorepellin proteins and their glycan variants have potential as biomarkers for the accurate diagnosis of pancreatic cystic lesions. In particular, the inventor has utilized a novel antibody-lectin sandwich microarray method to measure the protein expression and glycosylation of MUC5AC, endorepellin, and other proteins implicated in pancreatic neoplasia in cyst fluid samples. The detection of two glycan variants on MUC5AC and one glycan variant of endorepellin using the lectin wheat-germ agglutinin and blood group H antigen antibodies discriminated mucin-producing cystic tumors (mucinous cystic neoplasms and intraductal papillary mucinous neoplasms) from benign cystic lesions (serous cystadenomas and pseudocysts) with a 94% accuracy, 90% sensitivity at 100% specificity in a pre-validation set and 84% accuracy, (78% sensitivity and 100% specificity) in independent, blinded samples. This finding will allow for more accurate diagnosis of pancreatic cystic lesions in patients who will benefit from surgical intervention.

In one aspect of the present invention, methods are provided for diagnosing whether a pancreatic cyst in a subject is malignant, the method comprises, obtaining a pancreatic cyst fluid from a pancreatic cyst lesion of the subject, measuring the levels of MUC5AC and endorepellin glycoforms present in the pancreatic cyst fluid, comparing the levels of MUC5AC and endorepellin glycoforms to a control pancreatic cyst sample level of MUC5AC and endorepellin glycoforms; and diagnosing the pancreatic cyst lesion as malignant if the levels of the MUC5AC and endorepellin glycoforms are differentially expressed compared to the levels of the MUC5AC and endorepellin glycans present in control pancreatic cyst sample(s). If such elevation is found, the subject's pancreatic cyst lesion is diagnosed as a malignant pancreatic cyst.

In another aspect, a method for determining the malignant potential of a pancreatic cyst lesion from a subject is provided. In this method, a medical professional obtains a pancreatic cyst fluid sample from a pancreatic cyst lesion of the subject having or suspected of having pancreatic cancer. The fluid sample is then interrogated by measuring the levels of MUC5AC-WGA, MUC5AC-BGH and endorepellin-WGA glycoforms present in the pancreatic cyst fluid sample. Then the levels of MUC5AC-WGA, MUC5AC-BGH and endorepellin-WGA glycoforms present in the pancreatic cyst fluid sample are compared to a statistical threshold level for MUC5AC-WGA, MUC5AC-BGH and endorepellin-WGA glycoforms obtained from a comparable control non-malignant pancreatic cyst lesions. The subject's pancreatic cyst lesion is diagnosed as a malignant pancreatic cyst if two of the three levels of MUC5AC-WGA, MUC5AC-BGH and endorepellin-WGA glycoforms are higher in the subject's pancreatic cyst fluid sample than the levels of the two glycoforms obtained from comparable control non-malignant pancreatic cyst lesions.

In another aspect, kits are provided for performing the described methods of the present invention. For example, an illustrative kit may contain, a substrate for depositing a discrete sample specimen; at least one binding reagent, wherein the at least one or more binding reagents are operable to bind specifically to one or more glycoforms of MUC5AC and/or one or more glycoforms of endorepellin; and a detection reagent operable to identify a complex formed between the one or more binding reagents and the MUC5AC and/or endorepellin glycoforms. In one example, the one or more binding reagents include wheat-germ agglutinin and an antibody to the blood group H antigen.

### BRIEF DESCRIPTION OF THE DRAWINGS

FIGS. 1A-1B show protein and glycan detection on antibody arrays. FIG. 1A shows a graphical representation of an illustrative antibody capture assay wherein the proteins captured by an antibody array may be probed either with an antibody, to measure the abundance of the core protein, or a lectin, to measure the glycans on the captured proteins. FIG. 1B shows an illustrative graphical representation of an array containing multiple samples, wherein identical arrays may be run on a single microscope slide for high-throughput and low-volume sample processing. The probing of an array with anti-MUC5AC or anti-endorepellin shows signals only at the anti-MUC5AC or anti-endorepellin capture antibody spots, respectively, but probing the array with WGA shows signals at several capture antibodies. The brightest spots in each array (not boxed) are biotinylated, positive-control proteins.

FIG. 2 is a bar graph illustrating the frequency of expression of three biomarkers in neoplastic and benign pancreatic cysts. An exhaustive search for combinations of markers providing accurate discrimination of the patient groups resulted in a three-marker panel. An elevation of two or more of the three markers (as defined by the threshold for each marker) was observed in all of the mucinous cyst samples but in none of the non-mucinous cyst samples. The column labels indicate the diagnosis. An asterisk indicates measurements that were above their respective thresholds.

FIGS. 3A and 3B show graphical plots of quantified fluorescence values from the anti-MUC5AC (A) and anti-endorepellin (B) capture antibodies. FIG. 3A shows box plots representing anti-MUC5AC capture antibody with an anti-MUC5AC detection antibody (upper left) gives the MUC5AC protein levels; FIG. 3B shows box plots representing anti-endorepellin detection at the anti-endorepellin capture antibody gives the endorepellin protein levels (lower left). Detection by WGA and anti-BGH gives the respective glycan levels at each capture antibody. The boxes give the upper and lower quartiles, the vertical lines define the signal range, and the horizontal lines mark the median values. M, mucinous; NM, non-mucinous. Each point is the average signal from triplicate arrays for an individual sample, and results from 47 samples are shown.

FIGS. 4A and 4B show graphical representation of identified glycoforms distinguishing between neoplastic pancreatic cysts and benign pancreatic cysts. FIG. 4A shows a three biomarker panel was applied to 47 cyst fluid samples (30 mucinous, 17 non-mucinous). Each column represents results from a sample, and the first three rows indicate each marker value. A yellow square indicates that the marker exceeded the threshold, and a black square indicates it was below the threshold. The bottom row indicates the classification. If two or more markers were elevated, a sample was classified as mucinous (yellow square); otherwise it was classified as non-mucinous (black square). The sample columns were grouped by marker pattern (e.g. those with three elevations were grouped together) for clarity. FIG. 4B shows results of biomarker differentiation using a blinded set of samples. The biomarker panel was applied to 25 blinded cyst fluid samples (18 mucinous, 7 non-mucinous) that had not been used for marker development. Note the similarity in sample sub-groups in panels shown in FIGS. 4A and 4B.

FIG. 5 shows a tabulated representation of glycan distribution, type, and structural information in neoplastic pancreatic cysts and benign pancreatic cysts using mass-spectrometry.

FIGS. 6A and 6B show the relative amount of endorepellin protein levels in various cyst fluid samples. FIG. 6A shows a photomicrograph of an endorepellin western blot probing cyst fluid samples with anti-endorepellin antibody. FIG. 6B shows the relative total endorepellin levels for the same samples as determined using an anti-endorepellin sandwich assay.

FIGS. 7A-7D shows total CEA in pre-validation and validation samples. FIG. 7A shows a table providing detection of CEA in pre-validation samples using anti-CEA antibodies. FIG. 7B is a bar chart indicating relative concentrations of CEA in mucinous and non-mucinous pancreatic cyst fluid using an antibody capture assay. FIG. 7C is a bar chart of relative concentrations of CEA in mucinous and non-mucinous pancreatic cyst fluid validation samples using an antibody capture assay. The solid line represents the optimized cutoff determined in pre-validation and the dashed line represents the reported cutoff of. The overall accuracy for CEA is 81.8% (9/11) whereas the accuracy reaches 100% (11/11) with the three biomarker marker panel of the present invention. FIG. 7D shows a western blot photomicrograph of various cyst fluid samples probed using a panel of 3 CEA antibodies, illustrating variation in performance for discriminating mucinous from non-mucinous cysts.

FIGS. 8A and 8B show bar charts representing detection and relative quantification of glycan motifs present on the proteins MUC5AC. FIG. 8A is bar charts illustrating amounts of various glycan motifs present on MUC5AC obtained from pancreatic cyst fluid samples from mucinous and non-mucinous cysts. A subset of the cyst fluid samples from the pre-validation and validation sets were analyzed by ALSA to probe specific glycan motifs on captured MUC5AC. Each sample was probed with six different lectins (ECA, STL, WGA, GSL II, DSL and LEL), and the signals at the MUC5AC capture antibody are presented (in two separate graphs for clarity). Data from arrays incubated with PBS, instead of a sample, are shown as negative controls. ECA, STL, and GSL II show good elevations in the same samples as WGA, but DSL and LEL do not. FIG. 8B is a bar chart indicating the relative amounts of lectin binding to glycoproteins (fibronectin, laminin and haptoglobin) and in total cell lysates (pancreatic cell lines BXPC3 and Panc 1). BXPC3 and Panc1 were used as positive controls for lectin binding. The proteins and lysates were spotted in the microarrays at a concentration of 250 μg/mL, and the signals at each spot were quantified after detection with the indicated lectins. Each lectin has a unique pattern of binding to the glycoproteins and lysates, consistent with differences between the lectins in glycan-binding specificity and a lack of general, non-specific binding. In all panels, each column is the average of three replicate arrays, and the error bars are the standard deviations.

These figures are provided by way of example and are not intended to limit the scope of the invention.

### DETAILED DESCRIPTION

### Definitions

For purposes of this disclosure, unless defined otherwise, technical and scientific terms used herein have the same meaning as commonly understood by one of ordinary skill in the art to which this invention belongs. See, e.g. Singleton et al., Dictionary of Microbiology and Molecular Biology 2nd ed., J. Wiley & Sons (New York, N.Y. 1994); Sambrook et al., Molecular Cloning, A Laboratory Manual, Cold Spring Harbor Press (Cold Spring Harbor, N.Y. 1989). These references are hereby incorporated into this disclosure by reference in their entireties.

Before the present methods and kits are described, it is to be understood that any invention is not limited to the particular processes, compositions, or methodologies described, as these may vary. Moreover, the processes, compositions, and methodologies described in particular embodiments are interchangeable. Therefore, for example, a composition, dosage regimen, route of administration, and so on described in a particular embodiment may be used in any of the methods described in other particular embodiments. It is also to be understood that the terminology used in the description is for the purpose of describing the particular versions or embodiments only, and is not intended to limit the scope of the present invention, which will be limited only by the appended claims. Unless clearly defined otherwise, all technical and scientific terms used herein have the same meanings as commonly understood by one of ordinary skill in the art. Although any methods similar or equivalent to those described herein can be used in the practice or testing of embodiments of the present invention, the preferred methods are now described. All publications and references mentioned herein are incorporated by reference. Nothing herein is to be construed as an admission that the invention is not entitled to antedate such disclosure by virtue of prior invention.

As used herein, and in the appended claims, the singular forms “a”, “an”, and “the” include plural reference unless the context clearly dictates otherwise.

“Detect” and “detecting” refer to identifying the presence, absence or amount of the object to be detected.

By the term “detectable moiety” is meant, for the purposes of the specification or claims, a label molecule (isotopic or non-isotopic) which is incorporated indirectly or directly into another molecule, wherein the label molecule facilitates the detection of the molecule in which it is incorporated. Thus, “detectable moiety” is used synonymously with “label molecule”. Synthesis of labeled molecules can be accomplished by any one of several methods known to those skilled in the art. Label molecules, known to those skilled in the art as being useful for detection, include chemiluminescent, fluorescent or luminescent molecules. Various fluorescent molecules are known in the art which are suitable for incorporation as labels for the methods of the present invention. The protocol for such incorporation may vary depending upon the fluorescent molecule

As used herein, “diagnosis” or “diagnosing” whether a pancreatic cyst is malignant includes the initial detection of a pancreatic cancer or a confirmation of a diagnosis of the disease that has been made from other signs and/or symptoms. A “diagnosis” can include a diagnosis of increased risk or potential that the pancreatic cyst will become malignant. A diagnosis may include a “prognosis,” that is, a future prediction of the progression of a benign pancreatic cyst to a malignant pancreatic cyst, based on the glycoform levels of MUC5AC and endorepellin in the biological sample. A diagnosis or prognosis may be based on one or more samplings of pancreatic cyst fluid from a subject.

The phrase “differentially present” refers to a difference in the quantity and/or the frequency of a protein(s), a polypeptide(s), a glycan alteration(s), or a carbohydrate epitope(s) present in samples taken from pancreatic cystic lesions having malignant potential as compared to samples taken from pancreatic cystic lesions having no malignant potential. For example, a protein(s), a polypeptide(s), a glycan alteration(s), or a carbohydrate epitope(s) may be differentially present in that it is present at an elevated level in samples from pancreatic cystic lesions having malignant potential as compared to samples from pancreatic cystic lesions having no malignant potential. A protein(s), a polypeptide(s), a glycan alteration(s), or a carbohydrate epitope(s) can be differentially present in terms of quantity, frequency or both. For the purpose of this invention, a protein(s), a polypeptide(s), a glycan alteration(s), or a carbohydrate epitope(s) is differentially present when there is at least an about a two-fold, preferably at least about a four-fold, more preferably at least about a six-fold, most preferably at least about a tenfold difference between the quantity and/or frequency of a given protein(s), polypeptide(s), glycan alteration(s), or carbohydrate epitope(s) in pancreatic cystic lesions having malignant potential as compared to pancreatic cyst lesions having no malignant potential.

A glycan generally refers to a carbohydrate polymer comprising N and/or O-glycosidic linkages of monosaccharides to form polysaccharides and oligosaccharides.

A “glycan alteration” means a change in glycosylation state in a protein or polypeptide including, but not limited to, an addition, deletion, substitution, truncation, branching, or chain extension of a carbohydrate group.

A “glycoform” refers to a glycoprotein having a particular glycan composition and/or configuration. For example, glycoforms of the present invention can include the glycoproteins MUC5AC and endorepellin, wherein the glycan of MUC5AC, for example, can be recognized by specific binding to a particular lectin, for example, wheat germ agglutinin or an antibody to the blood group H antigen. The glycoform of endorepellin can include endorepellin having a glycan composition and/or configuration that specifically binds to the lectin wheat-germ agglutinin. The glycoforms of the present invention can be readily identified using an antibody capture lectin assay (ACLS) wherein specific proteins out of biological solutions are immobilized with a specific antibody, and lectins or glycan-specific antibodies detect the glycans present on the captured proteins.

A tissue has “malignant potential” if that tissue is likely to progress to cancer or already is cancerous. For example, a pancreatic cyst has malignant potential if that cyst is likely to develop into a mucinous cystic neoplasm (MCN) or an intraductal papillary mucinous neoplasm (IPMN).

“Pancreatic cyst fluid sample” means any fluid derived from a cystic lesion of the pancreas of a subject.

As used herein, a “subject” or “patient” is a warm blooded mammal, including, humans, farm animals such as horses, sheep, cattle, lamas, pigs and the like, as well as pets such as cats and dogs. In one embodiment, the warm blooded mammal is a human.

As used herein, the term “about” means plus or minus 10% of the numerical value of the number with which it is being used. Therefore, about 50% means in the range of 45%-55%.

“Optional” or “optionally” may be taken to mean that the subsequently described structure, event or circumstance may or may not occur, and that the description includes both instances where the event occurs and instances where it does not.

“Administering” or “administered”, when used in conjunction with a treatment or a therapeutic, means to administer a treatment or a therapeutic directly to, into or onto a target tissue or to administer a treatment or a therapeutic to a subject whereby the treatment or therapeutic positively impacts the tissue to which it is targeted. “Administering” a composition may be accomplished by oral administration, injection, infusion, absorption or by any method in combination with other known techniques. “Administering” may include the act of self-administration or administration by another person such as a healthcare provider or a device. As used herein, the term “administration” refers to the act of giving or administering a therapeutic treatment (e.g., therapeutic agents for the treatment of pancreatic cancer) to a subject (e.g., a subject or in vivo, in vitro, or ex vivo cells, tissues, and organs). Exemplary routes of administration to the human body can be through the eyes (ophthalmic), mouth (oral), skin (transdermal), nose (nasal), lungs (inhalant), oral mucosa (buccal), ear, by injection (e.g., intravenously, subcutaneously, intramuscularly, intratumorally, intraperitoneally, etc.) and the like.

The term “amino acid” not only encompasses the 20 common amino acids in naturally synthesized proteins, but also includes any modified, unusual, or synthetic amino acid. One of ordinary skill in the art would be familiar with modified, unusual, or synthetic amino acids.

The term “improves” is used to convey that the present invention refers to the overall physical state of an individual to whom an active agent has been administered. For example, the overall physical state of an individual may “improve” if one or more symptoms of a neurodegenerative disorder are alleviated by administration of an active agent. “Improves may also refer to changes in the appearance, form, characteristics, and/or physical attributes of tissue, or any combination thereof, to which it is being provided, applied, or administered.

As used herein, the term “therapeutic” means an agent utilized to treat, combat, ameliorate, or prevent, or any combination thereof, an unwanted condition or disease of a subject.

As used herein, the term “effective amount” refers to the amount of a composition sufficient to effect beneficial or desired results. An effective amount can be administered in one or more administrations, applications or dosages and is not intended to be limited to a particular formulation or administration route. An effective amount may include a therapeutically effective amount, or a non-therapeutically effective amount.

The terms “therapeutically effective amount” or “therapeutic dose” as used herein are interchangeable and may refer to the amount of an active agent or pharmaceutical compound or composition that elicits a biological and/or medicinal response in a tissue, system, animal, individual or human that is being sought by a researcher, veterinarian, medical doctor or other clinician, or any combination thereof. A biological or medicinal response may include, for example, one or more of the following: (1) preventing a disorder, disease, or condition in an individual that may be predisposed to the disorder, disease, or condition but does not yet experience or display pathology or symptoms of the disorder, disease, or condition, (2) inhibiting a disorder, disease, or condition in an individual that is experiencing or displaying the pathology or symptoms of the disorder, disease, or condition or arresting further development of the pathology and/or symptoms of the disorder, disease, or condition, and/or (3) ameliorating a disorder, disease, or condition in an individual that is experiencing or exhibiting the pathology or symptoms of the disorder, disease, or condition or reversing the pathology and/or symptoms disorder, disease, or condition experienced or exhibited by the individual.

The term “treatment” or “treating” as used herein refers to the administration of a therapeutic agent or the performance of a medical or surgical procedure with respect to a subject in need thereof, for either prophylaxis (prevention) or to cure or reduce the extent of or likelihood of occurrence or recurrence of the infirmity or malady or condition or event in the instance where the subject is afflicted. The term “treating” may also be taken to mean prophylaxis of a specific disorder, disease, or condition, alleviation of the symptoms associated with a specific disorder, disease, or condition and/or prevention of the symptoms associated with a specific disorder, disease or condition. In some embodiments, the term refers to slowing the progression of the disorder, disease, or condition or alleviating the symptoms associated with the specific disorder, disease, or condition. In some embodiments, the term refers to alleviating the symptoms associated with the specific disorder, disease, or condition. In some embodiments, the term refers to restoring function which was impaired or lost due to a specific disorder, disease, or condition. As related to the present invention, the term may also mean the administration of medicine or the performance of a medical procedure as therapy, prevention or prophylaxis of pancreatic cancer, e.g., the surgical removal of a pre-malignant precursor lesion or administration of radiation therapy.

As used herein, “protein” is a polymer consisting essentially of any of the 20 amino acids. Although “polypeptide” is often used in reference to relatively large polypeptides, and “peptide” is often used in reference to small polypeptides, usage of these terms in the art overlaps and is varied. The terms “peptide(s)”, “protein(s)” and “polypeptide(s)” are used interchangeably herein.

The term “wild-type” or “native” (used interchangeably) refers to the naturally-occurring polynucleotide sequence encoding a protein, or a portion thereof, or protein sequence, or portion thereof, respectively, as it normally exists in vivo.

The term “isolated” or “purified” polypeptide as used herein refers to a polypeptide that has been separated or purified from cellular components that naturally accompany it. Typically, the polypeptide is considered “purified” when it is at least 70% (e.g., at least 75%, 80%, 85%, 90%, 95%, or 99%) by dry weight, free from the proteins and naturally occurring molecules with which it is naturally associated.

As used herein, the term “subject diagnosed with a malignant pancreatic cyst” refers to a subject who has been tested and diagnosed to have a malignant pancreatic cyst, for example, mucinous cystic neoplasms (MCN) and intraductal papillary mucinous neoplasms (IPMN) which are precursors to pancreatic adenocarcinomas.

One novel strategy for the development of biomarkers for pancreatic cancer is to analyze carbohydrate alterations associated with particular proteins found in the cyst fluid of a subject having or suspected of having a malignant cyst. Changes to glycans on proteins are common in pancreatic cancer and are thought to play functional roles in the disease. The detection of carbohydrate changes may yield more effective biomarkers relative to measurements of core protein levels because they may be altered more reliably.

### A. Mucinous Pancreatic Cyst Biomarkers

In various embodiments, the present invention provides the discovery of prognostic and diagnostic biomarkers and methods of their use to determine whether a pancreatic cyst in a subject may develop or has developed into a malignant mucinous cystic neoplasm or an intraductal papillary mucinous neoplasm, collectively referred to as malignant pancreatic cysts. In certain aspects, the invention provides diagnostic and prognostic assays and methods to discriminate cystic pancreatic tumors from benign cystic lesions. That is, the present methods and assays may be used to diagnose, prognose, and treat a cancerous pancreatic lesion in a subject known or suspected of having pancreatic cystic lesion. The inventor has identified several specific glycoforms, including two derived from the mucin glycoprotein MUC5AC and one derived from the glycoprotein endorepellin. These glycoforms can be specifically identified with appropriate lectins and/or antibodies. In one embodiment, these glycoforms are characterized by specific binding to wheat-germ agglutinin (WGA) and a blood group H (BGH).

Mucins are high molecular weight glycoproteins that are predominantly produced by secretory epithelial cells. The membrane or secretory proteins are major constituents of the mucus layer that protects the gastric epithelium from mechanical and chemical assault. At least 14 genes have been identified as being involved in coding for the several MUC proteins. In some embodiments, mucin proteins are designated as MUC1, MUC2, MUC3, MUC4, MUC5A, MUC5B, MUC6, MUC7, MUC8, MUC9, MUC11, MUC12, MUC13, and MUC16. Mucins have a tandem-repeat domain rich in serine and threonine residues. These residues have numerous potential O-glycosylation sites for the attachment of β-glycan chains that make up to about 80% of the final molecular weight of the glycoprotein.

The present inventor has previously discovered that the detection of a glycan variant on MUC5AC using the lectin wheat-germ agglutinin discriminated mucin-producing cystic tumors (mucinous cystic neoplasms and intraductal papillary mucinous neoplasms) from benign cystic lesions (serous cystadenomas and pseudocysts) with a 78% sensitivity at 80% specificity, and when used in combination with cyst fluid CA 19-9 gave a sensitivity of 87% at 86% specificity, significantly better than the performance of CEA, as discussed in the International PCT Patent Application Publication No. WO 2011/082321 filed on Dec. 30, 2010 and incorporated herein by reference in its entirety.

A representative human amino acid sequence or wild-type human amino acid sequence of Mucin-5AC (MUC5AC) can be found in the National Center for Biotechnology Information (NCBI) databases as XM_{—}003403450.3, XP_{—}003403498.3, GI:410170618.

Endorepellin (also known as Perlecan domain V) is the C-terminal portion (amino acids 3,687-4,391) of the glycoprotein Perlecan (also known as Basement membrane-specific heparan sulfate proteoglycan core protein). Various in vitro and in vivo roles have been suggested for endorepellin, for example, a binding partner for endostatin and as an anti-angiogenic factor in VEGF-induced migration of HUVEC cells. In one embodiment, a representative endorepellin protein amino acid sequence can be found in the NCBI databases as (amino acids 3,687-4,391 of human Perlecan), Accession No. P98160, Version: P98160.4, GI:317373536.

A representative human amino acid sequence or wild-type human amino acid sequence of MUC5A and endorepellin is shown as amino acid sequences SEQ ID NO: 1 & 2 respectively in Table 1.

In some embodiments, a MUC5AC protein of the present invention can include a MUC5AC protein or fragment thereof, having at least about 80%, 85%, 90%, 91%, 92%, 93% 94%, 95%, 96%, 97%, 98% or 99% amino acid sequence identity with a MUC5AC sequence as disclosed herein, as provided in Table 1. Ordinarily, a MUC5AC protein will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to a MUC5AC protein sequence as disclosed herein, for example, the amino acid sequence of human MUC5AC protein of SEQ ID NO:1 as provided in Table 1.

In some embodiments, an endorepellin protein of the present invention can include a endorepellin protein or fragment thereof, having at least about 80%, 85%, 90%, 91%, 92%, 93% 94%, 95%, 96%, 97%, 98% or 99% amino acid sequence identity with a endorepellin sequence as disclosed herein, as provided in Table 1. Ordinarily, an endorepellin protein will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, 99%, or 99.5% or more amino acid sequence identity, to an endorepellin protein sequence as disclosed herein, for example, the amino acid sequence of human endorepellin protein of SEQ ID NO:2 as provided in table 1.

“Percent (%) amino acid sequence identity” with respect to a peptide or polypeptide sequence is defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2, as described in U.S. Pat. No. 6,828,146.

In some embodiments, exemplary MUC5AC and endorepellin protein amino acid sequences of SEQ ID NO:1 & 2 are provided in Table 1 below.

TABLE 1
MUC5A and Endorepellin polypeptides and nucleic acids.

| SEQ | | |
|---|---|---|
| ID | | |
| NO | Amino Acid or Nucleotide Sequence | Comment |
| 1 | MSLQFNGFVR CDHTVLPTEL NGLLGFPWTV GQAPGAHCGG | Wild-type Human |
| | SEMQGSEVLT CAPMPWLVWA PGNHSEGSTL WLVLSPRPTR | MUC5A |
| | MGIHGAHGLA GPPIREGQHV HSGAGGQGTS RQHPLTLMGL | XP_003403498.3 |
| | WGYQEHGLSE ARPGAVLDPT AHPGLGEVAL GHPRLFPQLC | |
| | GAEAIHRGLS PPDVCPPVCI HLSDPEERGA LAAAQDQSCG | |
| | ASRTRRLLRD RALFPAVHTM SVGRRKLALL WALALALACT | |
| | RHTGHAQDGS SESSYKHHPA LSPIARGPSG VPLRGATVFP | |
| | SLRTIPVVRA SNPAHNGRVC STWGSFHYKT FDGDVFRFPG | |
| | LCNYVFSEHC GAAYEDFNIQ LRRSQESAAP TLSRVLMKVD | |
| | GVVIQLTKGS VLVNGHPVLL PFSQSGVLIQ QSSSYTKVEA | |
| | RLGLVLMWNH DDSLLLELDT KYANKTCGLC GDFNGMPVVS | |
| | ELLSHNTKLT PMEFGNLQKM DDPTEQCQDP VPEPPRNCST | |
| | GFGICEELLH GQLFSGCVAL VDVGSYLEAC RQDLCFCEDT | |
| | DLLSCVCHTL AEYSRQCTHA GGLPQDWRGP DFCPQKCPNN | |
| | MQYHECRSPC ADTCSNQEHS RACEDHCVAG CFCPEGTVLD | |
| | DIGQTGCVPV SKCACVYNGA AYAPGATYST DCTNCTCSGG | |
| | RWSCQEVPCP GTCSVLGGAH FSTFDGKQYT VHGDCSYVLT | |
| | KPCDSSAFTV LAELRRCGLT DSETCLKSVT LSLDGVQTVV | |
| | VIKASGEVFL NQIYTQLPIS AANVTIFRPS TFFIIAQTSL | |
| | GLQLNLQLVP TMQLFMQLAP KLRGQTCGLC GNFNSIQADD | |
| | FRTLSGVVEA TAAAFFNTFK TQAACPNIRN SFEDPCSLSV | |
| | ENGTGVHGSP LCWPPGAGSP ASIWHCRQRG WPCVCL | |
| 2 | EIKITFRPDS ADGMLLYNGQ KRVPGSPTNL ANRQPDFISF | Wild-type human |
| | GLVGGRPEFR FDAGSGMATI RHPTPLALGH FHTVTLLRSL | endorepellin |
| | TQGSLIVGDL APVNGTSQGK FQGLDLNEEL YLGGYPDYGA | (amino acids 3687- |
| | IPKAGLSSGF IGCVRELRIQ GEEIVFHDLN LTAHGISHCP | 4391 of human |
| | TCRDRPCQNG GQCHDSESSS YVCVCPAGFT GSRCEHSQAL | Perlecan), |
| | HCHPEACGPD ATCVNRPDGR GYTCRCHLGR SGLRCEEGVT | Accession No. |
| | VTTPSLSGAG SYLALPALTN THHELRLDVE FKPLAPDGVL | P98160 |
| | LFSGGKSGPV EDFVSLAMVG GHLEFRYELG SGLAVLRSAE | Version: |
| | PLALGRWHRV SAERLNKDGS LRVNGGRPVL RSSPGKSQGL | P98160.4 |
| | NLHTLLYLGG VEPSVPLSPA TNMSAHFRGC VGEVSVNGKR | GI: 317373536 |
| | LDLTYSFLGS QGIGQCYDSS PCERQPCQHG ATCMPAGEYE | |
| | FQCLCRDGFK GDLCEHEENP CQLREPCLHG GTCQGTRCLC | |
| | LPGFSGPRCQ QGSGHGIAES DWHLEGSGGN DAPGQYGAYF | |
| | HDDGFLAFPG HVFSRSLPEV PETIELEVRT STASGLLLWQ | |
| | GVEVGEAGQG KDFISLGLQD GHLVFRYQLG SGEARLVSED | |
| | PINDGEWHRV TALREGRRGS IQVDGEELVS GRSPGPNVAV | |
| | NAKGSVYIGG APDVATLTGG RFSSGITGCV KNLVLHSARP | |
| | GAPPPQPLDL QHRAQAGANT RPCPS | |

### B. Methods of Diagnosing

In some embodiments, the present invention provides methods for diagnosing a cystic pancreatic tumor or malignancy in subject, and discriminates from benign cystic lesions. In one embodiment, present inventive method comprises:

a. obtaining a pancreatic cyst fluid sample from a pancreatic cyst lesion of the subject;

b. measuring the level of MUC5AC and endorepellin glycoforms present in the pancreatic cyst fluid sample;

c. comparing the levels of MUC5AC and endorepellin glycoforms to a control pancreatic cyst sample level of MUC5AC and endorepellin glycoforms; and

d. diagnosing the pancreatic cyst lesion as malignant if the levels of the MUC5AC and endorepellin glycoforms are differentially expressed as compared to the levels of the MUC5AC and endorepellin glycoforms present in control pancreatic cyst samples.

The inventive diagnostic and prognostic methods may be based upon the steps of: obtaining a pancreatic cyst fluid sample from a pancreatic cyst in a subject; contacting the sample with a glycan-binding protein, such as a lectin and/or an antibody, or fragment thereof (to detect the level of particular glycoforms of MUC5AC and endorepellin); detecting the levels of the particular glycoforms of MUC5AC and endorepellin in the sample; determining whether the MUC5AC and endorepellin glycoforms are differentially present in the sample, for example, by comparing the glycoform levels of each of MUC5AC and endorepellin in the sample with controls for these glycoforms of MUC5AC and endorepellin. For example, if the levels of two glycoforms of MUC5AC and one glycoform of endorepellin are elevated over control levels of these glycoforms of MUC5AC and endorepellin obtained from non-malignant pancreatic cysts, then the pancreatic cyst lesion is diagnosed as malignant or as having malignant potential.

In some embodiments of the present invention, a fluid sample of a pancreatic cyst is obtained from a subject who is being diagnosed or treated using the methods described herein. In one embodiment, a subject having a pancreatic cystic lesion or suspected of having a pancreatic cyst lesion can be initially screened using either computer tomography (CT) scanning or endosonography, or both, to initially locate and identify a pancreatic cyst lesion. In some embodiments, CT examination of the subject's pancreas can be performed using a dual-phase protocol performed to determine the presence of pancreatic lesions. In an illustrative embodiment, a CT scanner can include a single-detector helical scanner (HiSpeed CT/i; General Electric Medical Systems, Milwaukee, Wis.). The CT scanning procedure can include administration of an oral contrast material (for example, 175 mL iopamidol 300 [Isovue]; Bracco Diagnostics, Princeton, N.J.) using a power injector (Medrad, Pittsburgh, Pa.) at a rate of 4 mL/sec. Unenhanced images to locate the pancreas can be acquired using a 10-mm collimation with a 10-mm interval. Arterial phase imaging through the entire pancreas can be performed with a 15-sec delay after an IV administration of contrast material. Images obtained during the arterial phase can be helically acquired using a 3-mm collimation and may be prospectively reconstructed at 3-mm intervals with a 1:1 pitch. Portal venous phase images through the entire liver and pancreas are preferably acquired 30-120 sec after the commencement of N administration of contrast material using a 5-mm collimation and can be prospectively reconstructed at 5-mm intervals at a 1.5:1 pitch. In some embodiments, a CT scanning procedure can employ a dual-phase pancreatic protocol using a multidetector helical scanner (LightSpeed QX/i; General Electric Medical Systems). Unenhanced images can be obtained using a 5-mm collimation at 5-mm intervals and a table speed of 15 mm per revolution. With a delay of 30 sec after the IV administration of contrast material, images can routinely be acquired during the arterial phase using a 1.25-mm collimation at a 6:1 pitch, a 1.25-mm interval, and a table speed of 7.5 mm per revolution through the entire pancreas. Portal venous phase images may be acquired using a 5-mm collimation, a 5-mm interval, a 3:1 pitch, and a table speed of 15 mm per revolution after a 65-sec delay.

In some embodiments, a pancreatic cystic lesion may be identified using endosonography. In one illustrative example of a gastroenterologist-performed endosonography procedure, a scanner (model GF-UM 130; Olympus America, Lake Success, N.Y.) coupled with a 7.5- to 12.0-MHz radial array transducer and another scanner (model FG-36UX; Pentax Precision, Orangeburg, N.Y.) with a 5- to 7.5-MHz linear array transducer can be used to acquire images of the pancreas and determine the presence of a pancreatic cystic lesion. Using endosonographic guidance, a medical professional may insert a fine needle and extract the fluid contents of the cyst under diagnostic evaluation for further processing in accordance with the methods of the present invention. In some embodiments, a fluid sample of the pancreatic cyst can be obtained using endoscopic ultrasound (EUS)-guided fine needle aspiration (FNA), or any common biopsy method commonly used in the field.

In certain aspects, the invention provides methods and assays which may be used to diagnose, prognose, and treat a cancerous pancreatic lesion in a subject known or suspected of having pancreatic cystic lesion. The inventors have identified diagnostic glycoforms of MUC5AC and endorepellin, defined by reactivity with one or more lectins, for example, wheat-germ agglutinin (WGA) and one or more antibodies, for example, a blood group H (BGH) antibody. In some embodiments, exemplary glycoforms that can be used as diagnostic biomarkers are provided in Table 4 hereinbelow. In some embodiments, diagnostic glycoforms can include MUC5AC-WGA, MUC5AC-BGH, and endorepellin-WGA. These three glycoforms or biomarkers, were significantly elevated in malignant mucinous cysts, whereas the core protein levels were not significantly elevated. A three-marker panel based on these glycoforms distinguished mucinous from non-mucinous cysts with 94% accuracy (90% sensitivity, 100% specificity) in a pre-validation sample set (n=47) and with 84% accuracy (78% sensitivity, 100% specificity) in independent, blinded samples (n=25). Targeted lectin measurements and mass spectrometry analyses indicated that the higher WGA and BGH reactivity was due to short oligosaccharides terminating in GlcNAc or N-acetyl-lactosamine with occasional α-1,2-linked fucose. The non-mucinous cysts were characterized by non-fucosylated, short glycans with capping sialic acid.

In some embodiments, the inventive methods and assays detect glycan alterations on MUC5AC and endorepellin proteins found in a pancreatic cyst fluid sample. The two glycan alterations on MUC5AC (MUC5AC-WGA and MUC5AC-BGH) and one glycan alteration on endorepellin (endorepellin-WGA) are detected in the fluid sample from the subject. In some embodiments, when comparing the levels of the three glycoforms, the inventors have discovered that any two of the panel of three glycoforms (selected from MUC5AC-WGA, MUC5AC-BGH, and endorepellin-WGA) if elevated over levels of MUC5AC-WGA, MUC5AC-BGH, and endorepellin-WGA tested from control samples from non-malignant mucinous cysts, indicate malignant potential in the sampled pancreatic cyst from the tested subject. In some embodiments, an elevated level of (MUC5AC-WGA and MUC5AC-BGH), or (MUC5AC-WGA and endorepellin-WGA), or (MUC5AC-BGH and endorepellin-WGA) over control levels of these glycoforms from non-mucinous pancreatic cysts can be indicative that the subject's cyst fluid sample comes from a malignant pancreatic cyst.

In various embodiments, any assay that will detect MUC5AC and endorepellin glycan levels can be used, whether assayed individually (e.g., by sandwich ELISA or other methods known in the art), by high throughput methods (e.g., by using antibody arrays such as those described herein) or by structural analyses, such as chromatography processes coupled with mass spectrometry (MS) or nuclear magnetic resonance (NMR). Various detectable labels, such as biotin, can be utilized to detect the glycan binding protein that is bound to the glycan. Generally, suitable detectable labels include radioactive, fluorescent, fluorogenic, chromogenic, or other chemical labels. Useful radiolabels, which are detected by gamma counter, scintillation counter, or auto radiography include ³H, ¹²⁵I, ¹³¹I, ³⁵S and ¹⁴C. Common fluorescent labels include fluorescein, rhodamine, dansyl, phycoerythrin, phycocyanin, allophycocyanin, ophthaldehyde, and fluoroescamine. The fluorophor, such as the dansyl group, must be excited by light of a particular wavelength to fluoresce. The protein can also be labeled for detection using fluorescence-emitting metals such as ¹⁵²Eu, or others of the lanthanide series.

In one embodiment, glycan analysis can be performed using an antibody-lectin sandwich array (ALSA) as illustrated in FIG. 1A. Antibody arrays capture multiple, specific proteins out of biological solutions, and lectins or glycan-specific antibodies detect the glycans on the captured proteins. By running standard sandwich assays along with assays measuring protein glycosylation, glycan levels can be compared with the core protein levels to determine the relationship between protein abundance and glycosylation between test samples and control samples (See also FIGS. 1A and 1B). This approach is complementary to other carbohydrate analysis methods. For example, chromatography and mass spectrometry can provide detailed structural and compositional information. NMR analysis of the glycan from isolated glycoproteins can also be made to determine glycan structure. In one embodiment, the present methods can employ lectins and antibodies to specific glycans to provide measurements of specific sub-structures, rather than complete structural information, with high precision and over multiple samples.

In an illustrative embodiment, once the pancreatic cyst fluid sample (e.g., an aspirated pancreatic cyst fluid sample) has been obtained, the next step comprises measuring the level of various glycoforms present in the fluid sample as provided above. A glycan-binding protein or peptide ligand, such as a lectin, or a glycan binding antibody (for example a blood group H binding antibody) may be used to detect glycosylation levels of a target protein or proteins, for example MUC5AC and endorepellin.

The binding specificities of the lectins and glycan antibodies utilized in the present methods can provide insight into the nature of the altered glycans. Lectins include carbohydrate-binding proteins from many sources regardless of their ability to agglutinate cells. Lectins have been found in many organisms, including, plants, viruses, microorganisms and animals. Most known lectins are multimeric, with non-covalently associated subunits, and this multimeric structure gives lectins their ability to agglutinate cells or form precipitates with glycoconjugates similar to antigen-antibody interactions. A common characteristic of lectins is that they bind to specifically defined carbohydrate structures. Because of this specificity that each lectin has for a particular carbohydrate structure, even oligosaccharides with identical sugar compositions can be distinguished. Some lectins bind only structures with mannose or glucose residues, while others recognize only galactose residues. Some lectins bind only if a particular sugar is in a terminal non-reducing position in the oligosaccharide, while others bind sugars within the oligosaccharide chain. Further, some lectins do not discriminate when binding to a and b anomers, while other lectins require the correct anomeric structure and a specific sequence.

For example, the lectin Vicia villosa (VVL) has specificity for terminal galactosamine (GalNAc), and the increased binding of VVL on MUC5AC from mucin-producing cystic tumors may be due to truncation of O-glycans at the core GalNAc. GalNAc attached to the serine or threonine residue, referred to as the Tn antigen, has been frequently associated with pancreatic cancer and other cancers. The Jacalin lectin, which also showed high binding to MUC5AC from mucin-producing cystic tumors, can bind the Tn antigen as well as the related T antigen (Galb1,3GalNAc), which also is strongly associated with cancer. The lectin WGA binds N-acetylyglucosamine (GlcNAc) and other saccharides, for example, GlcNAcβ-4GlcNAcβ1-4GlcNAc, Neu5Ac (sialic acid). Increased GlcNAc could be due to increased branching of O-glycans or N-glycans, resulting in increased extension of glycan chains through repeated lactosamine (Gal131,4GlcNAc units. Both N-glycan branching and O-glycan branching are associated with the formation of cancer-associated glycans such as the Lewis blood group structures. The Lewis blood group structures are ligands for selectin receptors found on endothelial cells and lymphocytes, and increased presentation of this structure on pancreatic cells leads to increased metastasis and reduced survival in pancreatic cancer. The Erythrina cristagalli lectin (ECL), which showed high binding to both MUC5AC and CEACAM6 from mucinous cysts, also binds lactosamine, which is consistent with the results using WGA.

In one embodiment, the BGH glycan can be measured and quantified by using an antibody or lectin that specifically binds to the BGH trisaccharide: Fucα1,2Galα1-4GlcNAc-. Antibodies to the BGH glycan are commercially available, for example, VRW (Radnor, Pa. USA) under catalog no. 101983-330 (Blood Group H AB antigen antibody).

In some embodiments, the diagnostic methods of the present invention employ a comparison step, for example, comparing the levels of a panel of three biomarkers, for example, MUC5AC-WGA, MUC5AC-BGH, and endorepellin-WGA to a statistical threshold or levels of the same three biomarkers obtained from an appropriately matched non-malignant pancreatic cyst control sample. Once the comparison has been made, a difference in the level of two of the biomarkers (for example, MUC5AC-WGA and MUC5AC-BGH, or MUC5AC-WGA and endorepellin-WGA, or MUC5AC-BGH and endorepellin-WGA) when compared to a statistically validated threshold may indicate the malignant potential of the pancreatic cystic lesion in the subject. The statistically validated threshold may be based upon levels of biomarkers, in comparable samples obtained from a control population, e.g., the general population, or a select population of human subjects, such as subjects having pancreatic pseudocysts and serous cystadenomas which are non-malignant pancreatic cysts. Appropriately matched control samples can also include, control fluid samples obtained from pancreatic cyst lesions that were non-malignant and obtained from one or more of the following controls: the same sex, the same age quartile, the same racial background or any other scientifically validated controlled sample relative to the subject being diagnosed or treated. In one embodiment, the select population may be comprised of apparently healthy subjects. “Apparently healthy”, as used herein, means individuals (preferably from the same sex) who have not previously had any signs or symptoms indicating the presence of malignant pancreatic cancer, including mucinous cystic neoplasms (MCN) and intraductal papillary mucinous neoplasms (IPMN).

In some embodiments, the statistically validated threshold may be related to the value used to characterize the level of the biomarker obtained from the subject. Thus, if the level of the biomarker is an absolute value, then the control value is also based upon an absolute value. The statistically validated threshold can take a variety of forms. The statistically validated threshold can be a single cut-off value, such as a median or mean. The statistically validated threshold can be established based upon comparative groups such as where the risk in one defined group is double the risk in another defined group. The statistically validated threshold can be divided equally (or unequally) into groups, such as a low risk group, a medium risk group and a high-risk group, or into quadrants, the lowest quadrant being individuals with the lowest risk the highest quadrant being individuals with the highest risk, and the subject's risk of having pancreatic cancer or a predisposition to develop pancreatic cancer can be based upon which group his or her test value falls.

Statistically validated threshold of the biomarkers obtained, such as for example, mean levels, median levels, or “cut-off’ levels, may be established by assaying a large sample of individuals in the general population or the select population and using a statistical model such as the predictive value method for selecting a positivity criterion or receiver operator characteristic curve that defines optimum specificity (highest true negative rate) and sensitivity (highest true positive rate) as described in Knapp, R. G., and Miller, M. C. (1992). Clinical Epidemiology and Biostatistics. William and Wilkins, Harual Publishing Co. Malvern, Pa., which is specifically incorporated herein by reference. A “cutoff value” can be determined for each biomarker that is assayed.

In some embodiments, glycan levels of each select biomarker in the pancreatic cyst fluid sample may be compared to a single control value or to a range of control values. If the level of the biomarker in the sample is different than the statistically validated threshold, the test subject is at greater risk of developing or having pancreatic cancer than individuals with levels comparable to the statistically validated threshold. The extent of the difference between the subject's biomarker(s) levels and statistically validated threshold is also useful for characterizing the extent of the risk and thereby, determining which individuals would most greatly benefit from certain aggressive therapies. In those cases where the statistically validated threshold ranges are divided into a plurality of groups, such as the statistically validated threshold ranges for individuals at high risk, average risk and low risk, the comparison involves determining into which group the subject's level of the relevant risk predictor falls.

### C. Methods of Treatment

In some embodiments, the present diagnostic and prognostic assays and methods described herein can be used to determine if and when treatment should be administered to the subject, e.g., surgical removal of pre-malignant precursor lesions (that have not yet developed into invasive cancer) should and should not be undertaken for an individual subject. For example, individuals with levels of two of the three biomarkers (MUC5AC-WGA, MUC5AC-BGH and endorepellin-WGA glycoforms) that differ (for example, are elevated) from a statistically validated threshold, or that are in the higher tertile or quartile of a “normal range,” could be identified as those in need of medical treatment. Such medical treatments are known in the art.

In an illustrative method for treating a subject known or suspected of having a pancreatic cyst lesion, steps to implement this method include: obtaining a pancreatic cyst fluid sample from a pancreatic cystic lesion in a patient, assaying the sample for a glycan level of MUC5AC-WGA, MUC5AC-BGH and endorepellin-WGA; determining whether two of the three glycan levels of MUC5AC-WGA, MUC5AC-BGH and endorepellin-WGA in the sample are present at a higher level than the two of the three glycan levels of MUC5AC-WGA, MUC5AC-BGH and endorepellin-WGA in pancreatic cystic lesions having no malignant potential; and treating the pancreatic cystic lesion from the patient if the two of the three glycan levels of MUC5AC-WGA, MUC5AC-BGH and endorepellin-WGA in the sample is present at the higher level. Medical treatment of the pancreatic cystic lesion may include surgical removal or targeted radiation therapy of the pancreatic cystic lesion, or administration of a therapeutically effective amount of a chemotherapeutic agent to the subject.

In various embodiments, the glycan levels of MUC5AC and endorepellin may be assayed with a lectin, the lectin may be wheat-germ agglutinin (WGA), and the glycan level of MUC5AC also may be assayed with an antibody specifically directed to bind to the BGH antigen, and the pancreatic cyst fluid sample may be obtained by endoscopic ultrasound fine-needle aspiration

In one embodiment, a method for treating a pancreatic cystic lesion in a subject comprises: obtaining a pancreatic cyst fluid sample from a pancreatic cystic lesion in a patient, detecting or assaying a glycan level of MUC5AC-WGA, MUC5AC-BGH and endorepellin-WGA; comparing two of the three glycan levels of MUC5AC-WGA, MUC5AC-BGH and endorepellin-WGA in the sample to a statistically validated threshold for the two of the three glycan determined levels of MUC5AC-WGA, MUC5AC-BGH and endorepellin-WGA, which statistically validated threshold for the two of the three glycan determined levels of MUC5AC-WGA, MUC5AC-BGH and endorepellin-WGA is based on glycan levels in the two of the three glycan determined levels of MUC5AC-WGA, MUC5AC-BGH and endorepellin-WGA in comparable control samples from benign pancreatic cysts; and treating the pancreatic cystic lesion from the subject if the glycan levels of two of the three glycan levels of MUC5AC-WGA, MUC5AC-BGH and endorepellin-WGA in the sample is different than the statistically validated threshold. With this method, the glycan levels of MUC5AC and endorepellin may be assayed with a lectin, the lectin may be wheat-germ agglutinin (WGA), the glycan level of MUC5AC-BGH also may be assayed with an antibody specifically directed to bind to the BGH antigen, and the sample may be obtained by endoscopic ultrasound fine-needle aspiration. Treating the pancreatic cystic lesion may include surgical removal, targeted radiation therapy of the pancreatic cystic lesion, or administration of a chemotherapeutic regimen to the subject.

### D. Kits

Another embodiment of the present invention is a diagnostic kit for discriminating pancreatic cystic tumors from benign cystic lesions. In one embodiment, a biomarker panel or array (as described herein) is provided to distinguish a pancreatic cystic tumor from a benign cystic lesion. The inventive kit for differentiating cystic pancreatic tumors from benign cystic lesions may include a. a substrate for depositing a discrete sample specimen; b. at least one binding reagent, the at least one binding reagent operable to bind specifically to at least two glycoforms selected from MUC5AC glycoforms and an endorepellin glycoform present in the discrete sample specimen; and c. a detection reagent operable to identify a complex formed between the at least one binding reagent and the at least two glycoforms.

In another embodiment, the kit can include (a) a substrate with an antibody array having anti-MUC5AC and anti-endorepellin capture antibodies bound thereto, (b) a detection antibody to the BGH antigen and a detection antibody or glycan binding protein (lectin) to detect the levels of WGA in a sample; and optionally (c) one or more containers for such detection reagents. For example, the diagnostic kit could include WGA to detect a glycan variant on MUC5AC and a glycan variant of endorepellin, an antibody to detect BGH present on MUC5AC and instructions to use the kit, as an early stage screen to differentiate benign pancreatic cysts from pancreatic cysts that have the potential to progress to pancreatic cancer.

Diagnostic kits of the present invention can include any appropriate glycan binding protein. Some examples include either WGA or a glycan-binding antibody such as a BGH antigen-binding antibody, or other glycan binding antibodies described herein or otherwise known in the art. In another embodiment, the inventive diagnostic kit includes capture and detection antibodies (or other glycan binding proteins) to capture and detect MUC5AC and endorepellin glycan levels using sandwich ELISA, or other methods known in the art, to individually detect MUC5AC glycan levels and endorepellin glycan levels. The inventive kits may be used to perform the methods described herein.

Generally, the inventor formulated and tested the hypothesis that certain protein glycoforms are more abundant in mucinous cysts than in non-mucinous cysts. The present work confirmed that a WGA-reactive glycoform of MUC5AC is highly abundant in mucinous cysts. The results of the experiments performed demonstrated for the first time that high abundance of a BGH-reactive glycoform of MUC5AC exists in mucinous cysts. In addition, BGH-reactive and WGA-reactive glycoforms of endorepellin are abundant in mucinous cysts. A three-marker panel comprising these markers accurately distinguished mucinous from non-mucinous cysts in an expanded sample set and in blinded samples. Combined mass spectrometry and lectin analyses suggested that the higher WGA and BGH reactivity was due to short-chain oligosaccharides terminating in GlcNAc or lactosamine with occasional α1,2-linked fucose.

The key to the accuracy of the biomarker panel was the detection of glycoforms of specific proteins, as opposed to total protein levels or total glycan levels. The protein levels of MUC5AC and endorepellin, without respect to glycosylation, were not different between the cyst types (FIG. 3), nor were the total levels of any particular glycan, measured over all protein carriers. In contrast, specific glycoforms of MUC5AC and endorepellin showed very strong associations with mucinous cysts (FIG. 3). Furthermore, glycoform differences were not widespread among the various proteins tested but were largely restricted to MUC5AC and endorepellin. These findings support the concept that the glycosylation of specific proteins is dependent on context and can be highly associated with disease states. For that reason, biomarker assays based on detecting specific glycoforms may be more effective than conventional protein assays (37), as also demonstrated by previous work on glycoforms of alpha-fetoprotein (38), haptoglobin (39), human IgG (40), and MUC1 (41).

Endorepellin previously had not been proposed as a marker for pancreatic cancer, although a fragment of endorepellin, termed LG3, was identified as a potential serological biomarker for breast cancer (42). Endorepellin is a cleavage product from the C terminus of the matrix glycoprotein perlecan, a proteoglycan found in nearly all epithelial basement membranes. The N terminus of perlecan, heavily glycosylated with heparin sulfate, modulates diverse signaling events and could be pro-angiogenic in tumors (43). Endorepellin, on the other hand, may inhibit angiogenesis (44). A previous study showed that increased angiogenesis portends poor outcomes in IPMNs (44) so the balance between the pro- and anti-angiogenic activities of perlecan could have consequences for the development of tumors.

MUC5AC expression elevates in early-stage pancreatic neoplasias and is associated with a poor prognosis (3, 46-48). Its normal function is to regulate and protect the adult airway, stomach and ocular epithelia but it may also be involved in cancer progression (49). For example, MUC5AC expression may stimulate loss of cell-cell contacts and increased expression of factors promoting invasion (50). The effects of glycosylation on these processes are not known, but the fact that specific glycoforms of both MUC5AC and endorepellin were highly elevated in the potentially-malignant cysts suggests that the glycans influence their function.

The blood group H antigen consists of fucose alpha-linked to the 2′ carbon of a terminal galactose. People who cannot make the H antigen, due to inactivating germline mutations in the FUT2 gene, are at increased risk for certain infections and autoimmune diseases of the gut (51), suggesting that the H antigen and related structures may modulate bacterial colonization of the gut. In addition, the H antigen may be higher in induced pluripotent stem cells (52) and embryonic stem cells (53) relative to fully differentiated cells. The production of such structures in potentially malignant cysts could indicate changes in differentiation state of the neoplastic epithelial cells.

The terminal GlcNAc structure, also found in this study to be highly abundant in mucinous cysts, may be unregulated in the tissue of several different types of carcinoma (54). One reason for the upregulation of this structure may be protection against pathogenic bacteria, as terminal, alpha-linked GlcNAc in the gut protects from invasion by H. pylori (55). Both WGA and GSL-II bind alpha-linked as well as beta-linked GlcNAc (as assessed by glycan array analysis), so the alpha-liked structure could be present on MUC5AC and endorepellin. A link between pancreatic cysts and intestinal functions are further suggested by the physical connection of IPMNs to the gut and the intestinal morphology adopted by some IPMNs (56). Terminal GlcNAc also could arise from incomplete glycosylation, with unknown consequences.

The practical value of these glycoforms may be an accurate biomarker panel for distinguishing mucinous from non-mucinous cysts. The three individual markers of the panel were elevated in distinct but overlapping groups of patients, so that their combination out-performed any individual marker. The repeated observation of the same sub-groups of marker elevation patterns (FIG. 4) supports the possibility that these patterns represent real subclasses. The performance of the panel (accuracy of 92%) was better than the CEA performances determined in this paper (accuracies of 62%-85%) and previous reports (accuracies of 55%-86% (3, 30, 31)). In any case, the marker panel shows promise for clinical application, since this level of performance, if validated, would exceed current methods and would provide a firm basis for clinical diagnosis when used in combination with standard methods.

The inventor has shown that specific protein glycoforms are highly associated with mucinous pancreatic cysts and that the combined measurement of three markers, MUC5AC-WGA, endorepellin-WGA and MUC5AC-BGH.

### EXAMPLES

### Example 1

### Materials and Methods

Cyst Fluid Samples.

The study was conducted in compliance with the guidelines of local Institutional Review Boards. Cyst fluid samples were collected at the University of Michigan Medical Center, Memorial Sloan-Kettering Cancer Center, the University of Arizona Medical Center, the University of Pittsburg Medical Center and Ospedale Sacro Cuore-Don Calabria Negrar, Italy (Table 2). All samples were collected by either endoscopic ultrasound-guided, fine-needle aspiration (EUS-FNA) or FNA from the surgically removed cysts. The specimens were kept on ice until aliquots were made and frozen at −80° C., within two hours of collection. The samples were sub-aliquotted, and an aliquot that had been thawed no more than twice was used for each experiment. Prior to each experiment, the cellular debris and clot fragments were removed from each aliquot by centrifuging for 10 minutes at 2,400×g and collecting the supernatant.

TABLE 2
Sample information.

| | | | | ¹Discovery | ¹Discovery | ¹Pre- | |
|---|---|---|---|---|---|---|---|
| ID | Diagnosis | Collection | Source | A | B | validation | ¹Validation |
| S03218 | Canc. | Surgical | U. Michigan | x | | y | |
| S03864 | Canc. | Surgical | U. Michigan | x | x | x | |
| S02578 | IPMN | Surgical | U. Michigan | x | x | x | |
| S03865 | IPMN | Surgical | U. Michigan | x | | x | |
| S03869 | IPMN | Surgical | U. Michigan | x | x | x | |
| S02572 | MCN | Surgical | U. Michigan | x | | x | |
| S02573 | MCN | Surgical | U. Michigan | x | x | x | |
| S02574 | MCN | Surgical | U. Michigan | x | | x | |
| S03871 | MCN | Surgical | U. Michigan | x | x | x | |
| S03872 | MCN | Surgical | U. Michigan | x | x | | |
| S03888 | MCN | EUS-FNA | U. Arizona | x | | | |
| S02576 | PC | Surgical | U. Michigan | x | x | x | |
| S02577 | PC | Surgical | U. Michigan | x | | x | |
| S03224 | PC | Surgical | U. Michigan | x | x | x | |
| S03225 | PC | Surgical | U. Michigan | x | | y | |
| S03866 | PC | Surgical | U. Michigan | x | | | |
| S02575 | SC | Surgical | U. Michigan | x | | x | |
| S02580 | SC | Surgical | U. Michigan | x | x | x | |
| S03867 | SC | Surgical | U. Michigan | x | x | x | |
| S03868 | SC | Surgical | U. Michigan | x | x | x | |
| S03884 | SC | EUS-FNA | U. Arizona | x | | | |
| S03886 | SC | EUS-FNA | U. Arizona | x | x | | |
| S03216 | Canc. | Surgical | U. Michigan | | x | y | |
| S03885 | Canc. | EUS-FNA | U. Arizona | | | x | |
| S03912 | Canc. | Surgical | MSKCC | | | x | |
| S02437 | IPMN | Surgical | U. Michigan | | | x | |
| S02438 | IPMN | Surgical | U. Michigan | | | x | |
| S02439 | IPMN | Surgical | U. Michigan | | | y | |
| S03209 | IPMN | Surgical | U. Michigan | | x | x | |
| S03215 | IPMN | Surgical | U. Michigan | | | x | |
| S03217 | IPMN | Surgical | U. Michigan | | x | y | |
| S03873 | IPMN | Surgical | U. Michigan | | | y | |
| S03883 | IPMN | EUS-FNA | U. Arizona | | | x | |
| S03914 | IPMN | Surgical | MSKCC | | | x | |
| S03916 | IPMN | Surgical | MSKCC | | | x | |
| S03922 | IPMN | Surgical | MSKCC | | | x | |
| S03923 | IPMN | Surgical | MSKCC | | | x | |
| S03926 | IPMN | Surgical | MSKCC | | | x | |
| S03931 | IPMN | Surgical | U. Michigan | | | y | |
| S04560 | IPMN | Surgical | U. Michigan | | | | x |
| S04561 | IPMN | Surgical | U. Michigan | | | | x |
| S04567 | IPMN | Surgical | U. Michigan | | | | x |
| S04574 | IPMN | Surgical | U. Michigan | | | | x |
| S04576 | IPMN | Surgical | U. Michigan | | | | x |
| S04580 | IPMN | Surgical | U. Michigan | | | | x |
| S04586 | IPMN | Surgical | U. Michigan | | | | x |
| S04588 | IPMN | Surgical | U. Michigan | | | | x |
| S02433 | MCN | Surgical | U. Michigan | | | y | |
| S02434 | MCN | Surgical | U. Michigan | | | x | |
| S02440 | MCN | Surgical | U. Michigan | | | x | |
| S02441 | MCN | Surgical | U. Michigan | | | x | |
| S02579 | MCN | Surgical | U. Michigan | | x | x | |
| S03211 | MCN | Surgical | U. Michigan | | x | y | |
| S03219 | MCN | Surgical | U. Michigan | | | x | |
| S03220 | MCN | Surgical | U. Michigan | | x | y | |
| S03221 | MCN | Surgical | U. Michigan | | | x | |
| S03930 | MCN | Surgical | U. Michigan | | | x | |
| S04572 | MCN | Surgical | U. Michigan | | | | x |
| S04582 | MCN | Surgical | U. Michigan | | | | x |
| S04584 | MCN | Surgical | U. Michigan | | | | x |
| S04585 | MCN | Surgical | U. Michigan | | | | x |
| S04589 | MCN | Surgical | U. Michigan | | | | x |
| S04590 | MCN | Surgical | U. Michigan | | | | x |
| S02442 | PC | Surgical | U. Michigan | | | x | |
| S02443 | PC | Surgical | U. Michigan | | | x | |
| S03212 | PC | Surgical | U. Michigan | | | x | |
| S03213 | PC | Surgical | U. Michigan | | x | x | |
| S03214 | PC | Surgical | U. Michigan | | x | x | |
| S04562 | PDAC | Surgical | U. Michigan | | | | x |
| S03210 | PEN | Surgical | U. Michigan | | | x | |
| S03877 | PEN | EUS-FNA | U. Arizona | | | x | |
| S03917 | PEN | Surgical | MSKCC | | | x | |
| S04559 | PEN | Surgical | U. Michigan | | | | x |
| S04566 | PEN | Surgical | U. Michigan | | | | x |
| S04578 | PEN | Surgical | U. Michigan | | | | x |
| S02436 | SC | Surgical | U. Michigan | | | x | |
| S02444 | SC | Surgical | U. Michigan | | | x | |
| S02445 | SC | Surgical | U. Michigan | | | x | |
| S03222 | SC | Surgical | U. Michigan | | x | x | |
| S03223 | SC | Surgical | U. Michigan | | x | x | |
| S04563 | SC | Surgical | U. Michigan | | | | x |
| S04564 | SC | Surgical | U. Michigan | | | | x |
| S04565 | SC | Surgical | U. Michigan | | | | x |
| S04569 | SC | Surgical | U. Michigan | | | | x |
| S04570 | SC | Surgical | U. Michigan | | | | x |
| S04575 | SC | Surgical | U. Michigan | | | | x |
| S04577 | SC | Surgical | U. Michigan | | | | x |

¹For each experiment set, an “x” indicates the sample was included. A “y” indicates the samples that were not always included in pre-validation experiments due to low volume.

All patients enrolled in this study had surgery to remove the cystic lesion. The surgical pathology report was used to confirm the diagnosis of cyst type in all patients. The mucinous cysts, with malignant potential, included intraductal papillary mucinous neoplasms (IPMN), mucinous cystic neoplasms (MCN), and cysts that developed in association with a cancerous mass from adenocarcinoma or neuroendocrine neoplasms. The non-mucinous cysts included serous cystadenomas (SC) and pseudocysts (PC).

Western Blot.

The cyst fluid samples were diluted 1:1 in sample buffer consisting of Laemmli Sample Buffer (Bio-Rad, Hercules, Calif.) and 2-mercaptoethanol while being heated for 5 mins at 100° C. Fifteen micro liters of each sample was loaded per lane onto precast 4% to 12% Bis-Tris SDS-PAGE gels (Criterion XT, Bio-rad, Hercules, Calif.). After electrophoresis, the separated proteins were transferred on to a 0.2 μm PVDF membrane (Sequi-blot, Bio-Rad, Hercules, Calif.) using Trans Blot® SD Semi-Dry Transfer Cell (Bio-Rad, Hercules, Calif.). The PVDF membrane was blocked with 3% BSA for 1 hour at room temperature. Detection was done by two-1 hour sequential incubation with the primary biotinylated anti-endorepellin antibody (Table 3, 1:3000 dilution) and the secondary horse radish peroxidase (HRP) conjugated streptavidin (Thermal Scientific, Rockford, Ill., 1:10000 dilution). The membrane was washed in PBST0.05 between detection steps and developed with Super Signal West Pico Chemiluminescent Substrate (Thermo Scientific, Rockford, Ill.) according to manufacturer's instructions. After staining, the membrane was visualized by Gel Doc XR+System (Bio-Rad, Hercules, Calif.).

Antibodies and Lectins.

The antibodies and lectins were purchased from various sources (Table 3). The capture antibodies to be printed onto microarray slides were purified by dialysis (Slide-A-Lyzer, Pierce Biotechnology, Rockford, Ill.) to phosphate buffered saline (PBS) and ultracentrifuged. Antibody or lectin was biotinylated with the EZ-Link-sulfo-NHS-LC-Biotin kit (Pierce Biotechnology, Rockford, Ill.) according to the manufacturer's instructions.

Biological Reagents.

The buffers and biological solutions used in microarray assays include: PBST0.5 or 0.1 (1×PBS+0.5% or 0.1% Tween-20), 10× sample buffer (1×PBS+1% Tween-20+1% Brij-35 (Thermo Scientific, Rockford, Ill.)), 4× IgG blocking cocktail (400 μg/ml each for mouse, sheep, and goat IgG, 800 μg/mL rabbit IgG in 1×PBS, antibodies from Jackson Immunoresearch), 10× protease inhibitor (Complete Tablet, Roche Applied Science, Indianapolis, Ind.) and 2× sample dilution buffer (2× sample buffer+2× protease inhibitor+2× IgG cocktail in 1×PBS).

Microarray Fabrication.

The capture antibodies (Table 3) were spotted to the protein microarray PATH® film slides (Grace Bio-Labs, Bend, Oreg.) which are coated with nitrocellulose. The antibody printing step was performed by the 2470 Arrayer Microarray Printing Platform (Aushon Biosystems, Billerica, Mass.). All antibodies that were used for printing are adjusted to 250 μg/ml. Each slide contains 48 identical arrays arranged in a 4×12 grid with a 4.5 mm spacing

TABLE 3
Biological reagent information.

| | | | | Discovery | Discovery | Pre- | |
|---|---|---|---|---|---|---|---|
| Name | Source | Clone ID | Catalog # | A | B | validation | Validation |
| Anti Blood Group | AbCam | 9A | ab20131 | x | | | |
| A (BGA) | | | | | | | |
| Anti Blood Group | AbCam | Z5H-2 | ab24224 | x | | | |
| B (BGB) | | | | | | | |
| Anti Blood Group | AbCam | 87-N | ab24222 | x | | x | x |
| H (BGH) | | | | | | | |
| Anti Lewis A (Ab2) | USBiological | 0.N.387 | L2052X | x | | | |
| Anti Lewis B | Thermo Scientific | 2-25LE | MA1- | x | | | |
| | | | 19346 | | | | |
| Anti Lewis X | AbCam | P12 | ab3358 | x | | | |
| Anti Lewis Y | USBiological | 8.S.289 | L2056 | x | | | |
| Anti Sialyl Lewis X | USBiological | 9L648 | S1013-51B | x | | | |
| Anti Sialyl Lewis A | Collaborator | NS19-9 | — | x | | | |
| Anti Sialyl Tn | Collaborator | B72.3 | — | x | | | |
| Mucin (STn) | | | | | | | |
| Anti LAcNAc | Collaborator | — | — | x | | | |
| Wheat Germ | Vector | — | B-1025 | x | x | x | x |
| Agglutinin (WGA) | Laboratories | | | | | | |
| Maackia Amurensis | Vector | — | L-1260 | | x | | |
| 2 (MAL2) | Laboratories | | | | | | |
| Ricinus Communis | Vector | — | L-1080 | | x | | |
| Agglutinin I (RCA) | Laboratories | | | | | | |
| Psophocarpus | Vector | — | B-1365 | | x | | |
| Tetragonolobus | Laboratories | | | | | | |
| Lectin (PTL I) | | | | | | | |
| Euonymus | Vector | — | B-1335 | | x | | |
| Europaeus Lectin | Laboratories | | | | | | |
| (EEL) | | | | | | | |
| Griffonia | Vector | — | BK-3000 | | | | |
| simplicifolia lectin | Laboratories | | | | | | |
| II (GSL II) | | | | | | | |
| Datura stramonium | Vector | — | BK-3000 | | | | |
| lectin (DSL) | Laboratories | | | | | | |
| Solanum tuberosum | Vector | — | BK-3000 | | | | |
| lectin (STL) | Laboratories | | | | | | |
| Lycopersicon | EY Laboratories | — | BA-7001-1 | | | | |
| esculentum Lectin | | | | | | | |
| (LEL) | | | | | | | |
| Erythrina Cristigalli | Vector | — | L-1140 | | | | |
| (ECA) | Laboratories | | | | | | |

Capture Antibodies/Glyco-conjugates

| | | | | | | | |
|---|---|---|---|---|---|---|---|
| Anti Von | DAKO | — | A0082 | x | | | |
| Willebrand Factor | | | | | | | |
| (VWF) | | | | | | | |
| Anti Tumor | Aarden (Holland) | — | — | x | | | |
| Necrosis Factor | | | | | | | |
| (TNF) | | | | | | | |
| Anti C-reactive | Sigma | — | C1688 | x | | | |
| Protein (CRP) | | | | | | | |
| Anti Interleukin 6 | Sigma | 6708.11 | I7901 | x | | | |
| (IL6) | | | | | | | |
| Anti Beta | Biotrend | J971118 | 5685-3010 | x | | | |
| Lipoprotein | | | | | | | |
| Anti IGFBP3 | R & D Systems | 84728.111 | MAB305 | x | | | |
| Anti MUC1 (Ab1) | USBiological | 1.B.831 | C0050-23 | x | x | | |
| Anti | USBiological | 2Q397 | C1299-94 | | x | x | |
| Carcinoembryonic | | | | | | | |
| Antigen (CEA) | | | | | | | |
| (Ab1) | | | | | | | |
| Anti β2 | USBiological | O.N.17 | M3890- | x | | | |
| Microglobulin | | | 05X | | | | |
| Anti Serum | AbCam | 115 | ab687 | x | | | |
| Amyloid A | | | | | | | |
| Anti Insulin-like | R&D Systems | — | AF-291- | x | x | x | |
| Growth Factor 1 | | | NA | | | | |
| (IGF1) | | | | | | | |
| Anti Glypican 3 | Santa Cruz | — | sc-11395 | x | | | |
| Anti Lewis A (Ab1) | AbCam | 7LE | ab3967 | | x | | |
| Anti Lewis X | AbCam | P12 | ab3358 | x | x | | |
| Anti Gamma- | American | — | 3570 | x | | | |
| Carboxyglutamyl | Diagnostica | | | | | | |
| Domain (Gla) | | | | | | | |
| Anti Glucagon | Lab Vision | — | RB-1422- | x | | | |
| | | | A1 | | | | |
| Anti Insulin | Lab Vision | I-10 | MS-1595- | x | | | |
| | | | PABX | | | | |
| Anti Matrix Gla | Alexis (Axxora) | 52.1C5D | ALX-804- | x | | | |
| Protein | | | 512 | | | | |
| Anti Epidermal | AbCam | EGF-10 | ab10409 | x | | | |
| Growth Factor | | | | | | | |
| (EGF) | | | | | | | |
| Anti TGFa | Lab Vision | MF9 | MS-670- | x | | | |
| | | | PABX | | | | |
| Anti Angiostatin | R & D Systems | 79735 | MAB926 | x | | | |
| Anti Angiogenin | Sigma | 14017.7 | A 9850 | x | | | |
| Anti PDGF B | R & D Systems | 108132 | MAB2201 | x | | | |
| Anti VEGF | R & D Systems | — | MAB293 | x | | | |
| Anti Interleukin 8 | R & D Systems | 6217 | MAB208 | x | | | |
| (IL8) | | | | | | | |
| Anti Endostatin | R & D Systems | — | DY1098 | x | | | |
| Anti Vitronectin | Biodesign | BDI215 | N77810M | x | | | |
| Anti MUC1 (Ab2) | AbCam | SM3 | ab22711 | x | | | |
| Anti Fibronectin | R & D Systems | — | AF1918 | x | x | x | |
| Anti Heparin | Haematologic | — | PAHCII-G | x | | | |
| Cofactor 2 | Tech. | | | | | | |
| Anti Interleukin 10 | PeproTech Inc. | — | 900-K21 | x | | | |
| (IL10) | | | | | | | |
| Anti Apolipoprotein | Calbiochem | — | 178422 | x | | | |
| A1 (APO A1) | | | | | | | |
| Anti CEACAM 6 | Santa Cruz | By114 | sc-20059 | | x | x | |
| Anti Laminin | USBiological | — | L1225-01A | x | | | |
| Anti Endorepellin | R & D Systems | — | AF2364 | x | | x | x |
| Anti Perlecan | USBiological | 3G166 | H1890-93 | x | | | |
| Anti MUC16 (Ab1) | AbCam | X325 | ab10033 | | x | x | |
| Anti MUC5AC | Biogenesis | 45M1 | 1695-0128 | x | x | x | x |
| (Ab1) | | | | | | | |
| Anti Interleukin 1β | R & D Systems | 8516 | MAB201 | x | | | |
| (IL1b) | | | | | | | |
| Anti Alpha 1 | AbCam | — | ab7633 | x | | | |
| Antitrypsin (A1AT) | | | | | | | |
| Anti Transforming | R & D Systems | 9016 | MAB240 | x | | | |
| Growth Factor β1 | | | | | | | |
| (TGFb1) | | | | | | | |
| Anti Human | Stemcell Tech. | 26G9C10 | 1350 | x | x | | |
| Erythropoietin | | | | | | | |
| (EPO-26) | | | | | | | |
| Anti Fibroblast | R & D Systems | 10043 | BAM233 | x | | | |
| Growth Factor β | | | | | | | |
| (FGFb) | | | | | | | |
| Anti Hepatocyte | R & D Systems | — | BAF294 | x | | | |
| Growth Factor | | | | | | | |
| (HGF) | | | | | | | |
| Anti Bradykinin | AbD Serotec | — | 0100-0443 | x | x | x | |
| Anti Galectin 1 | R & D Systems | 201002 | 965-CY | x | | | |
| Anti Galectin 3 | R & D Systems | — | AF1197 | x | | | |
| Anti Prostate | Fitzgerald | — | 20-pr50 | x | | | |
| Specific Antigen | | | | | | | |
| (PSA) | | | | | | | |
| Anti MUC2 (Ab1) | AbCam | 994/152 | ab22712 | x | | | |
| Anti MUC1 (Ab3) | GeneTex | CM1 | GTX10114 | | x | x | |
| Anti MUC16 (Ab2) | GeneTex | X75 | GTX10029 | x | | | |
| Anti MUC16 (Ab3) | Novus Biologicals | X306 | NB120- | | | x | |
| | | | 10032 | | | | |
| Anti MUC16 (Ab4) | USBiological | 1.B.826 | C0050-05 | x | | | |
| Anti Modified C- | Larry Potempa | 3H12 | — | x | | | |
| reactive Protein | | | | | | | |
| (mCRP) (+) | | | | | | | |
| Anti Lewis A (Ab2) | USBiological | 0.N.387 | L2052X | x | | | |
| Anti MUC3 | AbCam | M3.1 | ab24068 | x | x | | |
| Anti MUC17 | Collaborator | SN1139-2 | — | x | | x | |
| Anti MUC2 (Ab2) | AbCam | 994/152 | ab22712 | | x | x | |
| Anti MUC5B | Novus Biologicals | — | H00004587- | x | | | |
| | | | A01 | | | | |
| Anti MUC3A | Life Diagnostics | 3H2744 | LS-C16658 | x | x | x | |
| Anti Serum | AbCam | SAP-5 | ab13334 | x | | | |
| Amyloid P (SAP) | | | | | | | |
| Anti Kininogen | Collaborator | 2B5 | — | x | | | |
| Anti Vimentin | R & D Systems | — | AF2105 | x | | | |
| Anti Human Milk | Thermo Scientific | EDM45 | 030402G | x | | | |
| Fat Globule 1 | | | | | | | |
| (HMFG1) | | | | | | | |
| Anti MUC5AC | Affinity | 2-11M1 | MA1- | x | x | x | |
| (Ab2) | BioReagents | | 35704 | | | | |
| Anti Apolipoprotein | GeneTex | — | GTX27620 | x | | | |
| E (APO E) | | | | | | | |
| Anti Mucin and | Sigma | — | HPA009173 | x | | | |
| Cadherin-Like | | | | | | | |
| Protein (MUCDHL) | | | | | | | |
| Anti Bacillus | Collaborator | 10F5 | — | x | x | | |
| anthracis Protective | | | | | | | |
| Antigen | | | | | | | |
| Anti Influenza | Collaborator | — | — | | x | | |
| Hemagglutinin-bio | | | | | | | |
| Anti Mannitou | Collaborator | — | — | x | | | |
| Anti CA19-9 (Ab1) | USBiological | 9L426 | C0075-03A | x | x | x | |
| Anti Blood Group | AbCam | 0.BG.5 | ab31754 | x | x | | |
| H1 + Blood Group | | | | | | | |
| H2 (BGH1/2) | | | | | | | |
| Anti Laminin 5 | AbCam | P3H9-2 | ab78286 | x | | | |
| Anti Blood Group | AbCam | Z5H-2 | ab24224 | x | x | | |
| B (BGB) | | | | | | | |
| Anti Blood Group | AbCam | 9A | ab20131 | x | x | | |
| A (BGA) | | | | | | | |
| Anti Versican | AbCam | MM0600- | ab89934 | x | | | |
| | | 7D41 | | | | | |
| Anti Sialyl Lewis X | USBiological | 9L648 | S1013-51B | x | x | | |
| Anti CA19-9 (Ab2) | AbCam | 121SLE | ab3982 | | x | | |
| Anti Blood Group | AbCam | K21 | ab3352 | x | x | | |
| Precursor | | | | | | | |
| Anti Lewis B | Thermo Scientific | 2-25LE | MA1- | x | x | | |
| | | | 19346 | | | | |
| Anti Lewis Y | USBiological | 8.S.289 | L2056 | x | x | | |
| Anti Gamma | Collaborator | — | — | | | x | |
| Glutamyl | | | | | | | |
| Transpeptidase | | | | | | | |
| (GGT) | | | | | | | |
| BSAGalb14Fuca13 | V-Labs | PCDX200 | NGP0302 | | | x | |
| GlcNAcb | | | | | | | |
| BSAGalb14Fucac3 | V-Labs | HGDX2- | NGP0502 | | | x | |
| GlcNAcb13Galb14 | | 008 | | | | | |
| GlcNAcb | | | | | | | |
| BSAGalb14GlcNA | V-Labs | YGDX1020 | NGP1201 | | | x | |
| cb | | | | | | | |
| Anti MUC4 | Collaborator | M8G7 | — | | | x | |
| Anti | Chemicon | — | MAB425 | | | x | |
| Carcinoembryonic | | | | | | | |
| Antigen (CEA) | | | | | | | |
| (Ab2) | | | | | | | |
| Anti | AbCam | — | ab15987 | | | x | |
| Carcinoembryonic | | | | | | | |
| Antigen (CEA) | | | | | | | |
| (Ab3) | | | | | | | |
| Anti | AbCam | 26/3/13 | ab4451 | | | x | |
| Carcinoembryonic | | | | | | | |
| Antigen (CEA) | | | | | | | |
| (Ab4) | | | | | | | |

| | | | |
|---|---|---|---|
| Name | Source | Clone ID | Catalog # |

Purified Glycoproteins

| | | | |
|---|---|---|---|
| Laminin | Sigma-Aldrich | — | L-6274 |
| Fibronectin | Sigma-Aldrich | — | F-0895 |
| Haptoglobin (Mixed Phenotypes) | Calbiochem | — | 327022 |
| Carcinoembryonic Antigen (CEA) | Fitzgerald Industries | 11257 | 30-AC32 |

Fluorescent and Labeling Reagents

| | | | |
|---|---|---|---|
| Strepavidin, B-phycoerythrine conjugate | Invitrogen | — | S32350 |

between arrays, and each array has the same antibodies printed in six-replicate. A wax-based hydrophobic boarder was imprinted to define boundaries between the arrays (SlideImprinter, The Gel Company, San Francisco, Calif.). The printed slides were stored at 4° C. in a desiccated, vacuum-sealed slide box until use.

Microarray Assays.

The antibody microarray assays with PATH slide was adapted and modified from the protocol described previously (1). Briefly, cyst fluid samples were diluted with 2× sample dilution buffer and incubated at 4° C. overnight for IgG blocking with gentle agitation. Unless otherwise stated, all the following steps were conducted at room temperature. The next day, the PATH slides were blocked with 1% Bovine Serum Albumin (BSA, Fisher Scientific, Fair Lawn, N J) in PBST0.5 for 1 hour, washed in three changes of PBST0.5 for 3 min each, and dried by brief centrifugation at 160×g.

A total of 6 μl of overnight IgG-blocked cyst fluid sample was applied to each array and incubated for 1 hour. After sample incubation, the slides were washed three times in PBST0.1 and spin-dried. Then the captured antigens were detected for 1 hour with biotinylated antibodies or lectins (3 μg/ml) prepared in PBST0.1 buffer, followed by 1 hour incubation with the secondary streptavidin-phycoerythrin (2 μg/ml) with three washes between steps. Lastly, all the spin-dried slides were scanned for fluorescence at 532 nm using a microarray scanner (LS Reloaded, TECAN, NC). The resulting images were quantified and analyzed with the software GenePix Pro 5.0 (Molecular Devices, Sunnyvale, Calif.), using both automatic and manual spot finding features. The local background was subtracted from the median intensity of each spot. The quantified results for each image were further processed to remove any outlier from the six-replicate spots with Grubb's test using a custom script. The geometric mean was calculated from the replicate spots for each capture antibody.

Total Protein Quantification.

Total protein measurements of cyst fluid were done with a Micro BCA Protein Assay kit (Thermo Scientific, Rockford, Ill.), according to the manufacturer microplate protocol. 96-well, flat bottom, 0.4 mL well microplates (Nalge Nunc International, Rochester, N.Y.) were used for the assays. Absorbance in each well was measured with the SpectraMax M2 Microplate Spectrophotometer (Molecular Devices, Sunnyvale, Calif.). A BSA dilution series was fitted and the curve used to calculate protein concentration in the samples.

Glycomic analyses. N-linked glycans from total cyst fluid glycoproteins were released according to previously described methods (2, 3). Briefly, samples (˜125 μg protein, 20 μL total volume) were thawed, diluted 2-fold with 8 M urea, 25 mM ammonium bicarbonate (pH 7.8), and clarified by centrifugation at 4° C. at 12,000 g×20 min. Supernatant samples were reduced by addition of DTT to 10 mM for 30 min, alkylated by the addition of iodoacetamide to 25 mM for 30 min, and quenched by an additional 25 mM DTT. Samples were then diluted to <1 M urea with 25 mM ammonium bicarbonate (pH 7.8) and subjected to trypsinolysis (total reaction volume ˜400 μL). The reaction was stopped by the addition of 30% (v/v) acetic acid to reduce the pH to below 3. Tryptic peptides were adsorbed to and released from a Waters C18-SepPak (100 mg), using 50% acetonitrile/0.1% TFA, dried, resuspended in 25 mM ammonium bicarbonate, and treated with PNGase F. Peptides were removed by passage through a C18-SepPak. Glycans were recovered from the flow-through by adsorption to and release from an activated charcoal cartridge (Carbograph, Grace, Deerfield, Ill.), and dried by vacuum centrifugation. Glycans were permethylated in a mixture of dimethyl sulfoxide and iodomethane, and supplemented with 500 mM NaCl prior to extraction into CHCl₃ (4). The CHCl₃ layer was multiply extracted with water and dried as above. Samples were characterized by MALDI-TOF/TOF mass spectrometry with an Ultraflex II (Bruker Daltonics, Billerica, Mass.) operated in reflectron-positive ion mode. MS and tandem MS data were processed using flexAnalysis 2.0 (Bruker).

Statistical Analyses and Presentation.

The data were analyzed and prepared using Microsoft Office Excel, OriginPro 8 (OriginLab, Northampton, Mass.), and MedCalc 12.3.0.0 (MedCalc Software, Mariakerke, Belgium). The figures were prepared using Canvas XII (ACD Systems).

### Example 2

### Protein Glycoforms Associated with Mucin-Producing Cysts

To test whether mucinous and non-mucinous cysts produce different levels of particular protein glycoforms, the ALSA platform was used to survey multiple glycan levels on a variety of protein carriers captured from cyst fluid. Twenty-two cyst fluid samples were profiled (11 from mucinous cysts and 11 from non-mucinous cysts, Table 2) in two experiment sets, one involving 72 capture antibodies and 12 glycan-binding detection reagents, and the other involving 27 capture antibodies and five detection reagents (Table 3). Each combination of capture antibody and detection reagent forms a unique assay, producing ˜1000 (72*12+27*5=999) capture antibody-lectin measurements. The antibody arrays targeted ˜30 different proteins, including mucins, matrix components, and secreted glycoproteins, and the detection reagents probed a variety of glycan structures that mucinous cysts might elevate (based on previous analyses of pancreatic cysts and cancers), including modifications to N-acetyl-lactosamine (LacNAc), ABO blood group structures, and Lewis family glycans. The mucinous cysts comprised intraductal papillary mucinous neoplasms (IPMN) and mucinous cystic neoplasms (MCN), and the non-mucinous cysts comprised serous cystadenoma (SC) and pseudocysts (PC).

Several antibody-lectin measurements were significantly different between the mucinous and non-mucinous cysts (Table 4). The markers that best distinguished the groups were endorepellin detected with WGA (designated endorepellin-WGA) and MUC5AC-WGA (p=0.0045 and p=0.013, respectively), followed by bradykinin detected by anti-blood group H (bradykinin-BGH, p=0.015), MUC5AC-EEL (p=0.020), and eight other markers with p<0.05. None of these was significantly different between MCN and IPMN. The pancreatic cancer marker CA 19-9 was highly elevated in about 30% of the samples but was not significantly different between any of the groups.

TABLE 4
Individual marker results from the discovery experiments.

| | Mucinous vs. | | Mucinous vs. | Mucinous vs. |
|---|---|---|---|---|
| Markers | Non-mucinous | IPMN vs. MCN | PC | SC |
| Anti Endorepellin_WGA | 0.0045 | | 0.0019 | 0.012 |
| Anti MUC5AC (Ab1)_WGA | 0.013 | | 0.016 | 0.011 |
| Anti Bradykinin_BGH | 0.015 | | 0.030 | 0.011 |
| Anti MUC5AC (Ab1)_EEL | 0.020 | | 0.025 | 0.017 |
| Anti MUC5AC (Ab2)_WGA | 0.022 | | 0.022 | 0.022 |
| Anti MUC1 (Ab1)_PTL1 | 0.033 | | 0.049 | 0.039 |
| Anti EGF_Lewis A (Ab2) | 0.033 | | | |
| Anti Fibronectin_Lewis X | 0.034 | | 0.039 | |
| Anti Fibronectin_WGA | 0.036 | | 0.022 | |
| Anti SAP_STn | 0.038 | | | |
| Anti Laminin 5_WGA | 0.039 | | 0.017 | |
| Anti CEA (Ab1)_PTL1 | 0.044 | | | |
| Anti Perlecan_STn | | 0.0072 | | |
| Anti EPO-26_STn | | 0.0092 | | |
| Anti Laminin 5_STn | | 0.013 | | |
| Anti Angiogenin_STn | | 0.015 | | 0.048 |
| Anti Matrix Gla Protein_STn | | 0.016 | | |
| Anti Laminin 5_BGA | | 0.018 | | |
| Anti PSA_Lewis A (Ab2) | | 0.019 | | |
| Anti PSA_STn | | 0.023 | | |
| Anti Matrix Gla Protein_Lewis A | | 0.023 | | |
| (Ab2) | | | | |
| Anti Bradykinin_MAL2 | | 0.029 | | |
| Anti Laminin 5_LacNAc | | 0.031 | | |
| Anti Serum Amyloid A_Lewis B | | 0.031 | | |
| Anti Laminin 5_Lewis Y | | 0.037 | | |
| Anti MUC16 (Ab2)_STn | | 0.042 | | |
| Anti EPO-26_MAL2 | | <0.001 | | |
| Anti Galectin 3_Sialyl Lewis X | | | 0.0057 | |
| Anti Mannitou_BGA | | | 0.0092 | |
| Anti IGFBP3_Sialyl Lewis X | | | 0.021 | |
| Anti MUC5AC (Ab2)_Lewis A | | | 0.026 | |
| (Ab2) | | | | |
| Anti MUC16 (Ab2)_BGA | | | 0.030 | |
| Anti TNF_Lewis X | | | 0.031 | |
| Anti IL10_Lewis B | | | 0.036 | |
| Anti Fibronectin_EEL | | | 0.037 | |
| Anti MUC5AC (Ab1)_RCA | | | 0.038 | |
| Anti Angiogenin_WGA | | | 0.038 | |
| Anti IL10_WGA | | | 0.038 | |
| Anti Heparin Cofactor 2_WGA | | | 0.039 | |
| Anti CEACAM 6_PTL1 | | | 0.040 | |
| Anti SAP_BGA | | | 0.041 | |
| Anti Fibronectin_WGA | | | 0.043 | |
| Anti CEACAM 6_EEL | | | 0.043 | |
| Anti MUC5AC (Ab1)_Sialyl Lewis X | | | 0.046 | |
| Anti Beta Lipoprotein_Lewis Y | | | | 0.0025 |
| Anti Gla_STn | | | | 0.0080 |
| Anti MUC16 (Ab1)_RCA | | | | 0.019 |
| Anti MUC1 (Ab3)_PTL1 | | | | 0.024 |
| Anti EPO-26_Lewis A (Ab2) | | | | 0.029 |
| Anti IGF1_Lewis A (Ab2) | | | | 0.030 |
| Anti IL8_STn | | | | 0.031 |
| Anti MUC5AC (Ab1)_PTL1 | | | | 0.033 |
| Anti MUC1 (Ab2)_WGA | | | | 0.034 |
| Anti CEACAM 6_RCA | | | | 0.036 |
| Anti CEA (Ab1)_WGA | | | | 0.040 |
| Anti BGA_PTL1 | | | | 0.041 |
| Anti Gla_Lewis X | | | | 0.046 |
| Anti MUC1 (Ab3)_WGA | | | | 0.048 |

P values were calculated by Student's t-test for each marker between groups.
Significant P values (<0.05) are shown in bold.
Capture antibodies binding glycans (for example, anti-Lewis X) were removed.
Anti-FGF was removed due to evidence of non-specific binding.

Extremely variable total protein concentrations were previously observed among the cyst fluid samples, resulting probably from varying levels of blood contamination (28). Here, the total protein concentrations also were highly variable, ranging from <100 μg/mL to >70 mg/mL (Table 2). The mucinous cysts (IPMN and MCN) had lower protein concentrations than the non-mucinous cysts (PC+SC) (median 5.9 vs. 11 mg/mL, respectively) with weak significance (p=0.06), and the IPMNs had lower concentrations than the MCNs (median 3.7 vs. 7.7 mg/mL, p=0.02). Because the individual marker measurements had different trends than the total protein concentrations, it was concluded that the individual measurements were not affected by variability in serum contamination, consistent with our previous analyses (28).

Next certain combinations of markers were investigated to determine if they provided added discriminatory value over any individual measurement, as observed previously using the combination of MUC5AC-WGA with CA 19-9 (20). Then the thresholds defining high and low states for each marker were scanned by using custom software, and then exhaustively searched for combinations of markers providing differential classification between the mucinous and non-mucinous cysts. Endorepellin and MUC5AC, detected with either WGA or BGH, provided nearly all the discriminatory power. A three-marker panel consisting of MUC5AC-WGA, MUC5AC-BGH, and endorepellin-WGA showed clear differences between the groups. The samples that were elevated in any two of those markers all were of the mucinous type, and all the samples that were elevated in only one or fewer of the markers were of the non-mucinous type (FIG. 2).

### Example 3

### Testing and Characterization of a Diagnostic, Three-Marker Panel

Next to better characterize the patterns of elevations of the identified markers using a larger sample set (n=47, including the previously used 22 samples) was sought. As before, MUC5AC and endorepellin, detected with either WGA or BGH, were significantly different between the groups (Table 5 and FIGS. 3A and 3B). Other significant markers were bradykinin detected with either WGA or BGH, as before, and CEA detected with WGA but not BGH. MUC16-WGA was slightly lower in the mucinous cysts (p=0.033).

TABLE 5
Individual marker results from the pre-validation experiments.

| | Mucinous vs. | IPMN vs. | Mucinous vs. | Mucinous vs. |
|---|---|---|---|---|
| Markers | Non-mucinous | MCN | PC | SC |
| Anti CEA(Ab4)_WGA | 1.0E−03 | | 0.00100 | 0.0043 |
| Anti MUC5AC(Ab1)_WGA | 1.0E−03 | | 0.0037 | 0.001 |
| Anti MUC5AC (Ab1)_¹BGH2X | 0.0046 | | 0.001 | |
| Anti Bradykinin_WGA | 0.0081 | | 0.016 | |
| Anti Endorepellin_¹BGH2X | 0.0099 | 0.012 | 0.013 | 0.033 |
| Anti MUC5AC (Ab1)_²BGH4X | 0.027 | | 0.030 | |
| Anti MUC16 (Ab1)_WGA | 0.033 | | | 0.014 |
| Anti Endorepellin_²BGH4X | 0.038 | 0.033 | 0.039 | |
| Anti MUC5AC (Ab2)_¹BGH2X | 0.042 | | 0.001 | |
| Anti Endorepellin_WGA | 0.043 | | 0.001 | |
| Anti Bradykinin_²BGH4X | 0.045 | | | 0.042 |
| Anti Fibronectin_WGA | | | 0.0040 | 0.001 |
| Anti Bradykinin_¹BGH2X | | | 0.0070 | |
| Anti MUC16 (Ab3)_¹BGH2X | | | 0.018 | |
| Anti MUC5AC (Ab2)_²BGH4X | | | 0.019 | |
| Anti CEA (Ab2)_WGA | | | 0.023 | |
| Anti CEACAM 6_WGA | | | | 0.001 |

P values were calculated by Student's t-test for each marker between groups.
Significant P values (<0.05) are shown in bold.
¹“BGH2X” indicates that cyst fluid samples were 2-fold diluted in the antibody-lectin microarray with BGH detection.
²“BGH4X” indicates that cyst fluid samples were 4-fold diluted in the antibody-lectin microarray with BGH detection.

The elevations observed in the mucinous cyst samples could result either from a change in the glycosylation state of the protein or from a change in the protein abundance (or a combination of both). To better characterize this relationship, measurements of the relative protein abundances of MUC5AC and endorepellin using antibody sandwich assays were obtained. Previously the specificity of the anti-MUC5AC antibody was confirmed, and the specificity of the anti-endorepellin antibody using Western blots was confirmed (FIG. 6A). The MUC5AC protein levels were moderately higher in the mucinous cysts, but not nearly as elevated as the WGA-reactive and BGH-reactive glycoforms of MUC5AC (FIG. 3A). Likewise, endorepellin protein levels were not significantly different between the cyst types, but the WGA-reactive and BGH-reactive glycoforms were (FIG. 3B). Both the protein measurements and the glycoform measurements were largely in the linear ranges of the assays, and both had good reproducibilities in replicate experiments (not shown). These analyses indicate that glycosylation states, not just protein levels, are different between the cyst types.

The three-marker panel correctly identified 24/30 (80%) of the cases and 17/17 (100%) of the controls (FIG. 4A), for an accuracy of 87%, demonstrating good consistency with the initial evaluation (FIG. 2). The thresholds defining elevations for each marker had to be re-derived because of the small preliminary sample set, so the result is not a complete validation of the panel. But the result does show that the relationships identified in the initial sample set are present also in this larger sample set, which supports the potential future value of the panel.

The panel misclassified all three of the samples collected from cysts associated with pancreatic neuroendocrine tumors (PNETs), suggesting the molecular profiles of the PNET cyst fluids are distinct from those of the IPMNs and MCNs. Three of the false negative cases and none of the true negative cases had elevated endorepellin-WGA, indicating this marker may be highly specific for cancer. If the classification rule is modified, such that samples elevated in any two markers or in endorepellin-WGA are classified as “cases,” the sensitivity increases to 90% without a drop in specificity. Four of the non-mucinous samples had elevated MUC5AC-WGA, but the panel classified the samples as controls because neither of the other markers was elevated, showing the value of using a panel of markers.

The biomarker panel was further tested with an independent, blinded set of cyst fluid samples (n=25) that had not been used in previous experiments. It was determined that the threshold for each marker using data from selected, previously run samples. The marker panel had a performance of 72% sensitivity (13/18), 100% specificity (7/7) and 80% accuracy (20/25) (FIG. 4B). The patterns of marker elevation were the same as in the previous sample set; some samples were elevated in all three markers, some in two, and others in one or none. As before, the PNETs were not classified as cases, but endorepellin-WGA was elevated in a subset. Classification of samples elevated in only endorepellin-WGA, the sensitivity improved to 14/18 (78%). The patterns in the non-mucinous cyst samples also were the same as before, with a small number showing elevation in MUC5AC-WGA (and also MUC5AC-BGH in this case) but none showing elevation in two markers or in endorepellin-WGA alone. This consistency between the datasets suggests the existence of patient subgroups with distinct marker expression patterns.

This performance is superior to the previously reported performance of CEA, which varies widely across studies (accuracies of 55%-86%. Here, using our antibody-array assay, CEA discriminated the patient groups in the pre-validation samples (n=45) and a subset of validation samples (n=13) with accuracies of 69%-85%, depending on the capture antibody used. (FIGS. 7A-7D). Comparing performance on the prevalidation samples the CEA antibodies appear to have divergent binding to distinct CEA glycoforms, as revealed by Western blot comparisons (FIG. 7D), which may contribute to the variability observed between CEA assays.

### Example 4

### Analyses of the Glycan Motifs Using Targeted Lectin Comparisons

The nature of the glycan structures associated with mucinous cysts was investigated using analyses of the specificities of the lectins that bind those glycans. The main, known binding preferences of WGA are for N-acetylglucosamine that is not substituted at the 3′ carbon and, more weakly, sialic acid. A statistical analysis of glycan array data, however, showed a more complex binding pattern that was hard to describe in terms of simple structures but that included N-acetylgalactosamine in certain presentations (32). To get more information about the glycan structures that are elevated in the mucinous cysts, 10 of the samples were probed, five mucinous and five non-mucinous, with five additional lectins. Lectins were chosen with specificities that overlap but are distinct from WGA. The lectins GSL II, ECA, and STL showed binding to captured MUC5AC that was similar to WGA, but DSL and LEL bound weakly to the captured MUC5AC in all the cyst fluid samples (FIG. 8A). GSL II is highly specific to terminal GlcNAc, ECA to terminal Galβ1,4 and terminal LacNAc, and STL to internal or terminal LacNAc and terminal Galβ1,4. In contrast, DSL and LEL mainly target internal LacNAc. All the lectins were verified as functional and specific through the use of positive and negative control samples (FIG. 8B). These data suggest that MUC5AC displays increased terminal LacNAc and GlcNAc in the mucinous cysts, but not extended, poly-LacNAc structures.

A comparison of the BGH binding profile to the profiles from antibodies against various Lewis and ABO antigens (obtained in the discovery experiments) shows little correlation with any (not shown), indicating the increased BGH signals were not from general fucosylation resulting in a wide variety of fucosylated structures but rather were primarily from non-sialylated, terminal galactose bearing α1,2-linked fucose (the H antigen).

### Example 5

### Analyses of the Glycan Motifs Using Glycomics Profiling

Additional information was gathered using N-glycomic profiling of total glycoproteins by mass spectrometry. This analysis does not tell us about glycans on individual proteins, but it could give insights into the major glycosylation shifts between the sample types. N-linked glycans were isolated from five mucinous and two non-mucinous cyst fluid samples, and m/z values were determined using MALDI TOF/TOF mass spectrometry (MS). 105 glycan compositions (various combinations of hexose, N-acetylhexosamine, deoxyhexose, sialic acid, and PO₄/SO₄ residues) were detected. As enumerated in Table 6, cyst-to-cyst variation was striking compared to the relative conservation of profiles reported for human neutrophils, kidney and liver tissue, and sera samples (33-35). Nevertheless, systematic commonalities were also observed (FIG. 5). For mucinous cysts, these included structures (inferred by imposing biosynthetic rules on compositions) with terminal LacNAc or GlcNAc, short extensions, prevalent fucosylation, and lack of sialylation. In comparison, non-mucinous cyst structures often exhibited capping sialic acids on their LacNAc extensions, with an overall predicted structure profile reminiscent of serum glycoproteins. This observation correlates with their higher protein concentration consistent with potential serum infusion (vide supra). The average differences between mucinous and non-mucinous samples summarized in FIG. 5 correlate with increased binding of WGA and BGH Ab to MUC5AC and endorepellin described above, and both glycoproteins likely express both N- and O-glycans (36). Further studies are needed to determine whether lectin recognition is targeted to N- or O-glycans on these glycoproteins. Nevertheless, the correlation suggests that mucinous cyst epithelia express elevated activities of selected GlcNAc-, Gal-, and Fuc-transferases that modify their N-glycans and potentially O-glycans as well with distinct peripheral glycan substructures.

TABLE 6
Complete glycomics data generated by MALDI-TOF/TOF mass spectrometry.

| | | | | | | | | | | Serous | Serous | MCN-L | IPMN-L | IPMN-L | IPMN-M | IPMN-M | IPMN-H | IPMN-H |
|---|---|---|---|---|---|---|---|---|---|---|---|---|---|---|---|---|---|---|
| Serous | Muc | Diff. | Hex | HexNAc | Fuc | NeuAc | Annotation | #Serous | #Muc | 3867 | 3868 | 3871 | 4198 | 4179 | 4173 | 3865 | 4175 | 4201 |
| 0.00% | 1.20% | 1.20% | 3 | 2 | 1 | 0 | Hex3- | 0 | 1 | 0.00% | 0.00% | 0.00% | 0.00% | 0.00% | 8.41% | 0.00% | 0.00% | 0.00% |
| | | | | | | | HexNAc2- | | | | | | | | | | | |
| | | | | | | | Fuc1- | | | | | | | | | | | |
| 0.00% | 2.22% | 2.22% | 3 | 2 | 2 | 0 | Hex3- | 0 | 1 | 0.00% | 0.00% | 0.00% | 0.00% | 0.00% | 15.53% | 0.00% | 0.00% | 0.00% |
| | | | | | | | HexNAc2- | | | | | | | | | | | |
| | | | | | | | Fuc2- | | | | | | | | | | | |
| 0.00% | 0.00% | 0.00% | 4 | 2 | 1 | 0 | Hex4- | 0 | 0 | 0.00% | 0.00% | 0.00% | 0.00% | 0.00% | 0.00% | 0.00% | 0.00% | 0.00% |
| | | | | | | | HexNAc2- | | | | | | | | | | | |
| | | | | | | | Fuc1- | | | | | | | | | | | |
| 0.69% | 1.62% | 0.92% | 5 | 2 | 0 | 0 | Hex5- | 1 | 2 | 1.39% | 0.00% | 0.00% | 2.04% | 0.00% | 0.00% | 9.28% | 0.00% | 0.00% |
| | | | | | | | HexNAc2- | | | | | | | | | | | |
| 0.39% | 1.73% | 1.35% | 3 | 3 | 1 | 0 | Hex3- | 1 | 4 | 0.00% | 0.77% | 0.00% | 0.00% | 4.05% | 2.88% | 0.00% | 2.00% | 3.21% |
| | | | | | | | HexNAc3- | | | | | | | | | | | |
| | | | | | | | Fuc1- | | | | | | | | | | | |
| 0.39% | 0.00% | 0.39% | 4 | 3 | 0 | 0 | Hex4- | 1 | 0 | 0.78% | 0.00% | 0.00% | 0.00% | 0.00% | 0.00% | 0.00% | 0.00% | 0.00% |
| | | | | | | | HexNAc3- | | | | | | | | | | | |
| 0.00% | 0.79% | 0.79% | 3 | 2 | 3 | 0 | Hex3- | 0 | 1 | 0.00% | 0.00% | 0.00% | 0.00% | 0.00% | 5.53% | 0.00% | 0.00% | 0.00% |
| | | | | | | | HexNAc2- | | | | | | | | | | | |
| | | | | | | | Fuc3- | | | | | | | | | | | |
| 0.00% | 0.46% | 0.46% | 4 | 2 | 2 | 0 | Hex4- | 0 | 1 | 0.00% | 0.00% | 0.00% | 0.00% | 0.00% | 3.24% | 0.00% | 0.00% | 0.00% |
| | | | | | | | HexNAc2- | | | | | | | | | | | |
| | | | | | | | Fuc2- | | | | | | | | | | | |
| 0.97% | 0.00% | 0.97% | 3 | 3 | 2 | 0 | Hex3- | 1 | 0 | 0.00% | 1.93% | 0.00% | 0.00% | 0.00% | 0.00% | 0.00% | 0.00% | 0.00% |
| | | | | | | | HexNAc3- | | | | | | | | | | | |
| | | | | | | | Fuc2- | | | | | | | | | | | |
| 0.44% | 0.96% | 0.52% | 6 | 2 | 0 | 0 | Hex6- | 1 | 2 | 0.00% | 0.89% | 0.00% | 0.00% | 0.00% | 0.00% | 4.26% | 2.48% | 0.00% |
| | | | | | | | HexNAc2- | | | | | | | | | | | |
| 0.00% | 0.45% | 0.45% | 4 | 3 | 1 | 0 | Hex4- | 0 | 1 | 0.00% | 0.00% | 0.00% | 0.00% | 0.00% | 3.16% | 0.00% | 0.00% | 0.00% |
| | | | | | | | HexNAc3- | | | | | | | | | | | |
| | | | | | | | Fuc1- | | | | | | | | | | | |
| 1.03% | 1.62% | 0.59% | 5 | 3 | 0 | 0 | Hex5- | 2 | 4 | 1.28% | 0.79% | 0.00% | 3.10% | 3.01% | 0.00% | 0.00% | 2.23% | 3.02% |
| | | | | | | | HexNAc3- | | | | | | | | | | | |
| 0.58% | 2.45% | 1.86% | 3 | 4 | 1 | 0 | Hex3- | 1 | 5 | 0.00% | 1.16% | 0.00% | 1.97% | 5.26% | 2.85% | 3.77% | 3.28% | 0.00% |
| | | | | | | | HexNAc4- | | | | | | | | | | | |
| | | | | | | | Fuc1- | | | | | | | | | | | |
| 0.52% | 2.11% | 1.59% | 4 | 4 | 0 | 0 | Hex4- | 1 | 6 | 0.00% | 1.04% | 0.00% | 2.30% | 3.49% | 2.19% | 1.51% | 2.62% | 2.69% |
| | | | | | | | HexNAc4- | | | | | | | | | | | |
| 0.00% | 0.49% | 0.49% | 4 | 2 | 3 | 0 | Hex4- | 0 | 1 | 0.00% | 0.00% | 0.00% | 0.00% | 0.00% | 3.44% | 0.00% | 0.00% | 0.00% |
| | | | | | | | HexNAc2- | | | | | | | | | | | |
| | | | | | | | Fuc3- | | | | | | | | | | | |
| 0.00% | 0.81% | 0.81% | 3 | 5 | 0 | 0 | Hex3- | 0 | 3 | 0.00% | 0.00% | 0.00% | 1.93% | 0.00% | 0.00% | 1.48% | 2.26% | 0.00% |
| | | | | | | | HexNAc5- | | | | | | | | | | | |
| 0.00% | 1.30% | 1.30% | 3 | 3 | 3 | 0 | Hex3- | 0 | 3 | 0.00% | 0.00% | 0.00% | 0.00% | 2.73% | 2.53% | 0.00% | 0.00% | 3.88% |
| | | | | | | | HexNAc3- | | | | | | | | | | | |
| | | | | | | | Fuc3- | | | | | | | | | | | |
| 0.86% | 1.08% | 0.22% | 4 | 3 | 2 | 0 | Hex4- | 1 | 2 | 0.00% | 1.73% | 0.00% | 0.00% | 0.00% | 5.21% | 0.00% | 0.00% | 2.35% |
| | | | | | | | HexNAc3- | | | | | | | | | | | |
| | | | | | | | Fuc2- | | | | | | | | | | | |
| 0.95% | 0.77% | 0.17% | 7 | 2 | 0 | 0 | Hex7- | 2 | 3 | 1.10% | 0.80% | 0.00% | 1.61% | 0.00% | 0.00% | 2.09% | 1.72% | 0.00% |
| | | | | | | | HexNAc2- | | | | | | | | | | | |
| 1.13% | 0.32% | 0.81% | 5 | 3 | 1 | 0 | Hex5- | 2 | 1 | 1.38% | 0.87% | 0.00% | 0.00% | 0.00% | 2.22% | 0.00% | 0.00% | 0.00% |
| | | | | | | | HexNAc3- | | | | | | | | | | | |
| | | | | | | | Fuc1- | | | | | | | | | | | |
| 1.71% | 4.11% | 2.40% | 4 | 4 | 1 | 0 | Hex4- | 2 | 7 | 1.54% | 1.89% | 4.38% | 2.77% | 7.03% | 2.19% | 3.20% | 5.34% | 3.90% |
| | | | | | | | HexNAc4- | | | | | | | | | | | |
| | | | | | | | Fuc1- | | | | | | | | | | | |
| 1.21% | 11.74% | 10.53% | 5 | 4 | 0 | 0 | Hex5- | 2 | 7 | 1.00% | 1.43% | 4.66% | 17.45% | 24.73% | 5.22% | 1.87% | 16.94% | 11.32% |
| | | | | | | | HexNAc4- | | | | | | | | | | | |
| 0.00% | 0.00% | 0.00% | 4 | 2 | 4 | 0 | Hex4- | 0 | 0 | 0.00% | 0.00% | 0.00% | 0.00% | 0.00% | 0.00% | 0.00% | 0.00% | 0.00% |
| | | | | | | | HexNAc2- | | | | | | | | | | | |
| | | | | | | | Fuc4- | | | | | | | | | | | |
| 0.00% | 0.71% | 0.71% | 3 | 5 | 1 | 0 | Hex3- | 0 | 2 | 0.00% | 0.00% | 0.00% | 0.00% | 0.00% | 0.00% | 2.61% | 2.35% | 0.00% |
| | | | | | | | HexNAc5- | | | | | | | | | | | |
| | | | | | | | Fuc1- | | | | | | | | | | | |
| 0.00% | 0.00% | 0.00% | 4 | 5 | 0 | 0 | Hex4- | 0 | 0 | 0.00% | 0.00% | 0.00% | 0.00% | 0.00% | 0.00% | 0.00% | 0.00% | 0.00% |
| | | | | | | | HexNAc5- | | | | | | | | | | | |
| 0.00% | 0.00% | 0.00% | 3 | 3 | 4 | 0 | Hex3- | 0 | 0 | 0.00% | 0.00% | 0.00% | 0.00% | 0.00% | 0.00% | 0.00% | 0.00% | 0.00% |
| | | | | | | | HexNAc3- | | | | | | | | | | | |
| | | | | | | | Fuc4- | | | | | | | | | | | |
| 0.00% | 1.33% | 1.33% | 4 | 3 | 3 | 0 | Hex4- | 0 | 4 | 0.00% | 0.00% | 0.00% | 0.00% | 0.00% | 4.03% | 1.24% | 1.60% | 2.42% |
| | | | | | | | HexNAc3- | | | | | | | | | | | |
| | | | | | | | Fuc3- | | | | | | | | | | | |
| 0.74% | 0.52% | 0.22% | 8 | 2 | 0 | 0 | Hex8- | 2 | 2 | 0.73% | 0.75% | 0.00% | 0.00% | 0.00% | 0.00% | 1.86% | 1.75% | 0.00% |
| | | | | | | | HexNAc2- | | | | | | | | | | | |
| 0.00% | 0.42% | 0.42% | 6 | 3 | 1 | 0 | Hex6- | 0 | 1 | 0.00% | 0.00% | 2.91% | 0.00% | 0.00% | 0.00% | 0.00% | 0.00% | 0.00% |
| | | | | | | | HexNAc3- | | | | | | | | | | | |
| | | | | | | | Fuc1- | | | | | | | | | | | |
| 1.33% | 2.70% | 1.37% | 4 | 4 | 2 | 0 | Hex4- | 2 | 7 | 0.86% | 1.79% | 3.20% | 2.48% | 3.16% | 2.36% | 1.67% | 1.66% | 4.34% |
| | | | | | | | HexNAc4- | | | | | | | | | | | |
| | | | | | | | Fuc2- | | | | | | | | | | | |
| 1.03% | 6.18% | 5.15% | 5 | 4 | 1 | 0 | Hex5- | 2 | 7 | 0.85% | 1.21% | 16.59% | 4.35% | 6.08% | 2.37% | 2.54% | 7.56% | 3.80% |
| | | | | | | | HexNAc4- | | | | | | | | | | | |
| | | | | | | | Fuc1- | | | | | | | | | | | |
| 0.28% | 1.11% | 0.83% | 6 | 4 | 0 | 0 | Hex6- | 1 | 3 | 0.00% | 0.56% | 0.00% | 3.00% | 0.00% | 0.00% | 0.00% | 1.80% | 2.97% |
| | | | | | | | HexNAc4- | | | | | | | | | | | |
| 0.52% | 2.55% | 2.03% | 4 | 5 | 1 | 0 | Hex4- | 2 | 7 | 0.51% | 0.53% | 2.91% | 2.65% | 2.82% | 2.49% | 1.97% | 2.56% | 2.47% |
| | | | | | | | HexNAc5- | | | | | | | | | | | |
| | | | | | | | Fuc1- | | | | | | | | | | | |
| 0.53% | 1.19% | 0.66% | 5 | 5 | 0 | 0 | Hex5- | 2 | 3 | 0.60% | 0.47% | 0.00% | 3.10% | 3.12% | 0.00% | 0.00% | 2.10% | 0.00% |
| | | | | | | | HexNAc5- | | | | | | | | | | | |
| 0.00% | 0.37% | 0.37% | 4 | 3 | 4 | 0 | Hex4- | 0 | 1 | 0.00% | 0.00% | 0.00% | 0.00% | 0.00% | 2.56% | 0.00% | 0.00% | 0.00% |
| | | | | | | | HexNAc3- | | | | | | | | | | | |
| | | | | | | | Fuc4- | | | | | | | | | | | |
| 0.00% | 0.64% | 0.64% | 6 | 3 | 2 | 0 | Hex6- | 0 | 2 | 0.00% | 0.00% | 3.15% | 0.00% | 0.00% | 0.00% | 1.35% | 0.00% | 0.00% |
| | | | | | | | HexNAc3- | | | | | | | | | | | |
| | | | | | | | Fuc2- | | | | | | | | | | | |
| 0.00% | 1.35% | 1.35% | 4 | 4 | 3 | 0 | Hex4- | 0 | 4 | 0.00% | 0.00% | 0.00% | 0.00% | 2.97% | 2.09% | 1.24% | 0.00% | 3.14% |
| | | | | | | | HexNAc4- | | | | | | | | | | | |
| | | | | | | | Fuc3- | | | | | | | | | | | |
| 0.00% | 0.24% | 0.24% | 9 | 2 | 0 | 0 | Hex9- | 0 | 1 | 0.00% | 0.00% | 0.00% | 0.00% | 0.00% | 0.00% | 1.70% | 0.00% | 0.00% |
| | | | | | | | HexNAc2- | | | | | | | | | | | |
| 0.00% | 3.53% | 3.53% | 5 | 4 | 2 | 0 | Hex5- | 0 | 2 | 0.00% | 0.00% | 20.99% | 0.00% | 0.00% | 0.00% | 3.71% | 0.00% | 0.00% |
| | | | | | | | HexNAc4- | | | | | | | | | | | |
| | | | | | | | Fuc2- | | | | | | | | | | | |
| 9.35% | 4.60% | 4.75% | 5 | 4 | 0 | 1 | Hex5- | 2 | 5 | 7.74% | 10.97% | 0.00% | 2.96% | 3.92% | 0.00% | 5.03% | 16.97% | 3.32% |
| | | | | | | | HexNAc4- | | | | | | | | | | | |
| | | | | | | | NeuAc1- | | | | | | | | | | | |
| 0.00% | 0.34% | 0.34% | 6 | 4 | 1 | 0 | Hex6- | 0 | 1 | 0.00% | 0.00% | 0.00% | 0.00% | 0.00% | 0.00% | 0.00% | 2.41% | 0.00% |
| | | | | | | | HexNAc4- | | | | | | | | | | | |
| | | | | | | | Fuc1- | | | | | | | | | | | |
| 0.00% | 0.31% | 0.31% | 4 | 5 | 2 | 0 | Hex4- | 0 | 1 | 0.00% | 0.00% | 0.00% | 0.00% | 0.00% | 0.00% | 2.16% | 0.00% | 0.00% |
| | | | | | | | HexNAc5- | | | | | | | | | | | |
| | | | | | | | Fuc2- | | | | | | | | | | | |
| 0.00% | 0.70% | 0.70% | 7 | 4 | 0 | 0 | Hex7- | 0 | 2 | 0.00% | 0.00% | 0.00% | 0.00% | 0.00% | 0.00% | 0.00% | 1.85% | 3.03% |
| | | | | | | | HexNAc4- | | | | | | | | | | | |
| 0.00% | 2.75% | 2.75% | 5 | 5 | 1 | 0 | Hex5- | 0 | 5 | 0.00% | 0.00% | 3.15% | 4.99% | 3.70% | 0.00% | 0.00% | 3.70% | 3.70% |
| | | | | | | | HexNAc5- | | | | | | | | | | | |
| | | | | | | | Fuc1- | | | | | | | | | | | |
| 0.87% | 3.92% | 3.05% | 6 | 5 | 0 | 0 | Hex6- | 2 | 5 | 0.88% | 0.87% | 0.00% | 9.00% | 6.79% | 3.15% | 0.00% | 3.51% | 4.99% |
| | | | | | | | HexNAc5- | | | | | | | | | | | |
| 0.00% | 0.00% | 0.00% | 5 | 3 | 4 | 0 | Hex5- | 0 | 0 | 0.00% | 0.00% | 0.00% | 0.00% | 0.00% | 0.00% | 0.00% | 0.00% | 0.00% |
| | | | | | | | HexNAc3- | | | | | | | | | | | |
| | | | | | | | Fuc4- | | | | | | | | | | | |
| 0.00% | 0.61% | 0.61% | 4 | 4 | 4 | 0 | Hex4- | 0 | 1 | 0.00% | 0.00% | 0.00% | 0.00% | 0.00% | 0.00% | 0.00% | 0.00% | 4.26% |
| | | | | | | | HexNAc4- | | | | | | | | | | | |
| | | | | | | | Fuc4- | | | | | | | | | | | |
| 0.00% | 1.79% | 1.79% | 5 | 4 | 3 | 0 | Hex5- | 0 | 3 | 0.00% | 0.00% | 6.47% | 0.00% | 0.00% | 2.82% | 3.25% | 0.00% | 0.00% |
| | | | | | | | HexNAc4- | | | | | | | | | | | |
| | | | | | | | Fuc3- | | | | | | | | | | | |
| 0.00% | 0.00% | 0.00% | 6 | 4 | 2 | 0 | Hex6- | 0 | 0 | 0.00% | 0.00% | 0.00% | 0.00% | 0.00% | 0.00% | 0.00% | 0.00% | 0.00% |
| | | | | | | | HexNAc4- | | | | | | | | | | | |
| | | | | | | | Fuc2- | | | | | | | | | | | |
| 0.00% | 0.26% | 0.26% | 4 | 5 | 3 | 0 | Hex4- | 0 | 1 | 0.00% | 0.00% | 0.00% | 0.00% | 0.00% | 0.00% | 1.83% | 0.00% | 0.00% |
| | | | | | | | HexNAc5- | | | | | | | | | | | |
| | | | | | | | Fuc3- | | | | | | | | | | | |
| 1.56% | 2.54% | 0.98% | 5 | 5 | 2 | 0 | Hex5- | 2 | 7 | 1.29% | 1.83% | 2.93% | 3.46% | 2.70% | 2.02% | 1.53% | 1.55% | 3.60% |
| | | | | | | | HexNAc5- | | | | | | | | | | | |
| | | | | | | | Fuc2- | | | | | | | | | | | |
| 0.61% | 1.98% | 1.37% | 6 | 5 | 1 | 0 | Hex6- | 2 | 5 | 0.61% | 0.60% | 3.20% | 3.68% | 3.10% | 2.00% | 0.00% | 1.88% | 0.00% |
| | | | | | | | HexNAc5- | | | | | | | | | | | |
| | | | | | | | Fuc1- | | | | | | | | | | | |
| 0.00% | 0.55% | 0.55% | 7 | 5 | 0 | 0 | Hex7- | 0 | 2 | 0.00% | 0.00% | 0.00% | 0.00% | 0.00% | 0.00% | 0.00% | 1.52% | 2.35% |
| | | | | | | | HexNAc5- | | | | | | | | | | | |
| 0.00% | 0.00% | 0.00% | 6 | 3 | 4 | 0 | Hex6- | 0 | 0 | 0.00% | 0.00% | 0.00% | 0.00% | 0.00% | 0.00% | 0.00% | 0.00% | 0.00% |
| | | | | | | | HexNAc3- | | | | | | | | | | | |
| | | | | | | | Fuc4- | | | | | | | | | | | |
| 4.19% | 0.26% | 3.93% | 4 | 4 | 5 | 0 | Hex4- | 2 | 1 | 2.56% | 5.82% | 0.00% | 0.00% | 0.00% | 0.00% | 1.83% | 0.00% | 0.00% |
| | | | | | | | HexNAc4- | | | | | | | | | | | |
| | | | | | | | Fuc5- | | | | | | | | | | | |
| 0.00% | 0.00% | 0.00% | 6 | 3 | 0 | 2 | Hex6- | 0 | 0 | 0.00% | 0.00% | 0.00% | 0.00% | 0.00% | 0.00% | 0.00% | 0.00% | 0.00% |
| | | | | | | | HexNAc3- | | | | | | | | | | | |
| | | | | | | | NeuAc2- | | | | | | | | | | | |
| 0.00% | 0.63% | 0.63% | 5 | 4 | 4 | 0 | Hex5- | 0 | 2 | 0.00% | 0.00% | 0.00% | 0.00% | 0.00% | 2.56% | 1.84% | 0.00% | 0.00% |
| | | | | | | | HexNAc4- | | | | | | | | | | | |
| | | | | | | | Fuc4- | | | | | | | | | | | |
| 54.26% | 3.23% | 51.03% | 5 | 4 | 0 | 2 | Hex5- | 2 | 3 | 55.65% | 52.88% | 2.69% | 0.00% | 0.00% | 0.00% | 17.59% | 2.35% | 0.00% |
| | | | | | | | HexNAc4- | | | | | | | | | | | |
| | | | | | | | NeuAc2- | | | | | | | | | | | |
| 0.00% | 0.00% | 0.00% | 6 | 4 | 3 | 0 | Hex6- | 0 | 0 | 0.00% | 0.00% | 0.00% | 0.00% | 0.00% | 0.00% | 0.00% | 0.00% | 0.00% |
| | | | | | | | HexNAc4- | | | | | | | | | | | |
| | | | | | | | Fuc3- | | | | | | | | | | | |
| 1.86% | 1.07% | 0.79% | 5 | 5 | 3 | 0 | Hex5- | 2 | 4 | 1.53% | 2.18% | 0.00% | 1.93% | 0.00% | 0.00% | 2.20% | 1.04% | 2.33% |
| | | | | | | | HexNAc5- | | | | | | | | | | | |
| | | | | | | | Fuc3- | | | | | | | | | | | |
| 0.00% | 0.99% | 0.99% | 6 | 5 | 2 | 0 | Hex6- | 0 | 2 | 0.00% | 0.00% | 5.07% | 0.00% | 0.00% | 1.85% | 0.00% | 0.00% | 0.00% |
| | | | | | | | HexNAc5- | | | | | | | | | | | |
| | | | | | | | Fuc2- | | | | | | | | | | | |
| 1.48% | 0.40% | 1.09% | 7 | 6 | 0 | 0 | Hex7- | 2 | 2 | 1.87% | 1.10% | 0.00% | 0.00% | 0.00% | 0.00% | 1.62% | 1.16% | 0.00% |
| | | | | | | | HexNAc6- | | | | | | | | | | | |
| 0.00% | 1.48% | 1.48% | 6 | 4 | 4 | 0 | Hex6- | 0 | 3 | 0.00% | 0.00% | 0.00% | 4.16% | 3.10% | 0.00% | 0.00% | 0.00% | 3.07% |
| | | | | | | | HexNAc4- | | | | | | | | | | | |
| | | | | | | | Fuc4- | | | | | | | | | | | |
| 0.00% | 1.30% | 1.30% | 6 | 5 | 3 | 0 | Hex6- | 0 | 4 | 0.00% | 0.00% | 4.38% | 0.00% | 0.00% | 1.61% | 1.31% | 0.00% | 1.79% |
| | | | | | | | HexNAc5- | | | | | | | | | | | |
| | | | | | | | Fuc3- | | | | | | | | | | | |
| 0.00% | 0.82% | 0.82% | 6 | 6 | 2 | 0 | Hex6- | 0 | 3 | 0.00% | 0.00% | 0.00% | 2.08% | 1.94% | 0.00% | 0.00% | 0.00% | 1.71% |
| | | | | | | | HexNAc6- | | | | | | | | | | | |
| | | | | | | | Fuc2- | | | | | | | | | | | |
| 0.00% | 0.00% | 0.00% | 6 | 6 | 0 | 1 | Hex6- | 0 | 0 | 0.00% | 0.00% | 0.00% | 0.00% | 0.00% | 0.00% | 0.00% | 0.00% | 0.00% |
| | | | | | | | HexNAc6- | | | | | | | | | | | |
| | | | | | | | NeuAc1- | | | | | | | | | | | |
| 0.00% | 0.00% | 0.00% | 5 | 4 | 2 | 2 | Hex5- | 0 | 0 | 0.00% | 0.00% | 0.00% | 0.00% | 0.00% | 0.00% | 0.00% | 0.00% | 0.00% |
| | | | | | | | HexNAc4- | | | | | | | | | | | |
| | | | | | | | Fuc2- | | | | | | | | | | | |
| | | | | | | | NeuAc2- | | | | | | | | | | | |
| 0.16% | 1.20% | 1.04% | 7 | 6 | 1 | 0 | Hex7- | 1 | 4 | 0.32% | 0.00% | 2.45% | 1.88% | 0.00% | 2.00% | 0.00% | 0.00% | 2.05% |
| | | | | | | | HexNAc6- | | | | | | | | | | | |
| | | | | | | | Fuc1- | | | | | | | | | | | |
| 0.47% | 0.22% | 0.25% | 7 | 7 | 0 | 0 | Hex7- | 2 | 1 | 0.58% | 0.36% | 0.00% | 1.55% | 0.00% | 0.00% | 0.00% | 0.00% | 0.00% |
| | | | | | | | HexNAc7- | | | | | | | | | | | |
| 0.00% | 0.54% | 0.54% | 6 | 5 | 4 | 0 | Hex6- | 0 | 2 | 0.00% | 0.00% | 0.00% | 0.00% | 0.00% | 0.00% | 1.79% | 0.00% | 1.96% |
| | | | | | | | HexNAc5- | | | | | | | | | | | |
| | | | | | | | Fuc4- | | | | | | | | | | | |
| 1.44% | 0.00% | 1.44% | 6 | 5 | 0 | 2 | Hex6- | 2 | 0 | 1.78% | 1.10% | 0.00% | 0.00% | 0.00% | 0.00% | 0.00% | 0.00% | 0.00% |
| | | | | | | | HexNAc5- | | | | | | | | | | | |
| | | | | | | | NeuAc2- | | | | | | | | | | | |
| 0.00% | 0.18% | 0.18% | 5 | 6 | 4 | 0 | Hex5- | 0 | 1 | 0.00% | 0.00% | 0.00% | 0.00% | 0.00% | 0.00% | 1.26% | 0.00% | 0.00% |
| | | | | | | | HexNAc6- | | | | | | | | | | | |
| | | | | | | | Fuc4- | | | | | | | | | | | |
| 0.00% | 0.59% | 0.59% | 6 | 3 | 1 | 3 | Hex6- | 0 | 2 | 0.00% | 0.00% | 0.00% | 1.52% | 0.00% | 0.00% | 0.00% | 0.00% | 2.59% |
| | | | | | | | HexNAc3- | | | | | | | | | | | |
| | | | | | | | Fuc1- | | | | | | | | | | | |
| | | | | | | | NeuAc3- | | | | | | | | | | | |
| 0.00% | 0.00% | 0.00% | 6 | 6 | 3 | 0 | Hex6- | 0 | 0 | 0.00% | 0.00% | 0.00% | 0.00% | 0.00% | 0.00% | 0.00% | 0.00% | 0.00% |
| | | | | | | | HexNAc6- | | | | | | | | | | | |
| | | | | | | | Fuc3- | | | | | | | | | | | |
| 0.00% | 0.56% | 0.56% | 7 | 6 | 2 | 0 | Hex7- | 0 | 2 | 0.00% | 0.00% | 2.45% | 1.46% | 0.00% | 0.00% | 0.00% | 0.00% | 0.00% |
| | | | | | | | HexNAc6- | | | | | | | | | | | |
| | | | | | | | Fuc2- | | | | | | | | | | | |
| 0.40% | 0.13% | 0.27% | 6 | 5 | 1 | 2 | Hex6- | 2 | 1 | 0.50% | 0.30% | 0.00% | 0.00% | 0.00% | 0.00% | 0.00% | 0.92% | 0.00% |
| | | | | | | | HexNAc5- | | | | | | | | | | | |
| | | | | | | | Fuc1- | | | | | | | | | | | |
| | | | | | | | NeuAc2- | | | | | | | | | | | |
| 0.00% | 0.00% | 0.00% | 8 | 7 | 0 | 0 | Hex8- | 0 | 0 | 0.00% | 0.00% | 0.00% | 0.00% | 0.00% | 0.00% | 0.00% | 0.00% | 0.00% |
| | | | | | | | HexNAc7- | | | | | | | | | | | |
| 0.00% | 0.00% | 0.00% | 7 | 5 | 4 | 0 | Hex7- | 0 | 0 | 0.00% | 0.00% | 0.00% | 0.00% | 0.00% | 0.00% | 0.00% | 0.00% | 0.00% |
| | | | | | | | HexNAc5- | | | | | | | | | | | |
| | | | | | | | Fuc4- | | | | | | | | | | | |
| 0.14% | 0.12% | 0.01% | 5 | 6 | 3 | 1 | Hex5- | 1 | 1 | 0.27% | 0.00% | 0.00% | 0.00% | 0.00% | 0.00% | 0.86% | 0.00% | 0.00% |
| | | | | | | | HexNAc6- | | | | | | | | | | | |
| | | | | | | | Fuc3- | | | | | | | | | | | |
| | | | | | | | NeuAc1- | | | | | | | | | | | |
| 0.26% | 1.05% | 0.79% | 6 | 6 | 4 | 0 | Hex6- | 2 | 4 | 0.27% | 0.25% | 0.00% | 1.17% | 2.74% | 0.00% | 1.16% | 0.00% | 2.28% |
| | | | | | | | HexNAc6- | | | | | | | | | | | |
| | | | | | | | Fuc4- | | | | | | | | | | | |
| 0.00% | 1.00% | 1.00% | 7 | 6 | 3 | 0 | Hex7- | 0 | 3 | 0.00% | 0.00% | 4.64% | 1.50% | 0.00% | 0.00% | 0.00% | 0.89% | 0.00% |
| | | | | | | | HexNAc6- | | | | | | | | | | | |
| | | | | | | | Fuc3- | | | | | | | | | | | |
| 0.00% | 0.71% | 0.71% | 7 | 7 | 2 | 0 | Hex7- | 0 | 3 | 0.00% | 0.00% | 0.00% | 1.65% | 1.80% | 0.00% | 0.00% | 0.00% | 1.52% |
| | | | | | | | HexNAc7- | | | | | | | | | | | |
| | | | | | | | Fuc2- | | | | | | | | | | | |
| 0.00% | 0.00% | 0.00% | 8 | 7 | 1 | 0 | Hex8- | 0 | 0 | 0.00% | 0.00% | 0.00% | 0.00% | 0.00% | 0.00% | 0.00% | 0.00% | 0.00% |
| | | | | | | | HexNAc7- | | | | | | | | | | | |
| | | | | | | | Fuc1- | | | | | | | | | | | |
| 0.00% | 0.40% | 0.40% | 7 | 5 | 5 | 0 | Hex7- | 0 | 2 | 0.00% | 0.00% | 0.00% | 1.34% | 0.00% | 1.46% | 0.00% | 0.00% | 0.00% |
| | | | | | | | HexNAc5- | | | | | | | | | | | |
| | | | | | | | Fuc5- | | | | | | | | | | | |
| 2.72% | 0.28% | 2.44% | 6 | 5 | 0 | 3 | Hex6- | 2 | 1 | 3.87% | 1.57% | 0.00% | 0.00% | 0.00% | 0.00% | 1.97% | 0.00% | 0.00% |
| | | | | | | | HexNAc5- | | | | | | | | | | | |
| | | | | | | | NeuAc3- | | | | | | | | | | | |
| 0.39% | 0.57% | 0.18% | 6 | 6 | 5 | 0 | Hex6- | 2 | 3 | 0.49% | 0.29% | 0.00% | 0.88% | 0.00% | 0.00% | 1.19% | 0.00% | 1.92% |
| | | | | | | | HexNAc6- | | | | | | | | | | | |
| | | | | | | | Fuc5- | | | | | | | | | | | |
| 0.19% | 0.00% | 0.19% | 6 | 6 | 3 | 1 | Hex6- | 1 | 0 | 0.38% | 0.00% | 0.00% | 0.00% | 0.00% | 0.00% | 0.00% | 0.00% | 0.00% |
| | | | | | | | HexNAc6- | | | | | | | | | | | |
| | | | | | | | Fuc3- | | | | | | | | | | | |
| | | | | | | | NeuAc1- | | | | | | | | | | | |
| 0.22% | 0.00% | 0.22% | 7 | 6 | 4 | 0 | Hex7- | 1 | 0 | 0.43% | 0.00% | 0.00% | 0.00% | 0.00% | 0.00% | 0.00% | 0.00% | 0.00% |
| | | | | | | | HexNAc6- | | | | | | | | | | | |
| | | | | | | | Fuc4- | | | | | | | | | | | |
| 0.12% | 0.68% | 0.57% | 7 | 7 | 3 | 0 | Hex7- | 1 | 3 | 0.23% | 0.00% | 0.00% | 1.52% | 0.00% | 0.00% | 0.92% | 0.00% | 2.35% |
| | | | | | | | HexNAc7- | | | | | | | | | | | |
| | | | | | | | Fuc3- | | | | | | | | | | | |
| 0.65% | 0.20% | 0.45% | 6 | 5 | 1 | 3 | Hex6- | 2 | 1 | 1.00% | 0.31% | 0.00% | 0.00% | 0.00% | 0.00% | 1.40% | 0.00% | 0.00% |
| | | | | | | | 4exNAc5- | | | | | | | | | | | |
| | | | | | | | Fuc1- | | | | | | | | | | | |
| | | | | | | | NeuAc3- | | | | | | | | | | | |
| 0.00% | 0.00% | 0.00% | 6 | 6 | 4 | 1 | Hex6- | 0 | 0 | 0.00% | 0.00% | 0.00% | 0.00% | 0.00% | 0.00% | 0.00% | 0.00% | 0.00% |
| | | | | | | | HexNAc6- | | | | | | | | | | | |
| | | | | | | | Fuc4- | | | | | | | | | | | |
| | | | | | | | NeuAc1- | | | | | | | | | | | |
| 0.00% | 0.15% | 0.15% | 7 | 6 | 5 | 0 | Hex7- | 0 | 1 | 0.00% | 0.00% | 0.00% | 1.04% | 0.00% | 0.00% | 0.00% | 0.00% | 0.00% |
| | | | | | | | HexNAc6- | | | | | | | | | | | |
| | | | | | | | Fuc5- | | | | | | | | | | | |
| 0.00% | 0.00% | 0.00% | 7 | 7 | 4 | 0 | Hex7- | 0 | 0 | 0.00% | 0.00% | 0.00% | 0.00% | 0.00% | 0.00% | 0.00% | 0.00% | 0.00% |
| | | | | | | | HexNAc7- | | | | | | | | | | | |
| | | | | | | | Fuc4- | | | | | | | | | | | |
| 0.00% | 0.00% | 0.00% | 8 | 7 | 3 | 0 | Hex8- | 0 | 0 | 0.00% | 0.00% | 0.00% | 0.00% | 0.00% | 0.00% | 0.00% | 0.00% | 0.00% |
| | | | | | | | HexNAc7- | | | | | | | | | | | |
| | | | | | | | Fuc3- | | | | | | | | | | | |
| 0.17% | 0.38% | 0.21% | 6 | 6 | 5 | 1 | Hex6- | 1 | 2 | 0.34% | 0.00% | 0.00% | 0.87% | 1.76% | 0.00% | 0.00% | 0.00% | 0.00% |
| | | | | | | | HexNAc6- | | | | | | | | | | | |
| | | | | | | | Fuc5- | | | | | | | | | | | |
| | | | | | | | NeuAc1- | | | | | | | | | | | |
| 0.75% | 0.04% | 0.71% | 7 | 6 | 0 | 3 | Hex7- | 1 | 1 | 1.50% | 0.00% | 0.00% | 0.30% | 0.00% | 0.00% | 0.00% | 0.00% | 0.00% |
| | | | | | | | HexNAc6- | | | | | | | | | | | |
| | | | | | | | NeuAc3- | | | | | | | | | | | |
| 0.20% | 0.52% | 0.32% | 7 | 7 | 5 | 0 | Hex7- | 1 | 2 | 0.39% | 0.00% | 0.00% | 0.00% | 0.00% | 0.00% | 1.92% | 0.00% | 1.69% |
| | | | | | | | HexNAc7- | | | | | | | | | | | |
| | | | | | | | Fuc5- | | | | | | | | | | | |
| 0.00% | 0.29% | 0.29% | 8 | 7 | 4 | 0 | Hex8- | 0 | 1 | 0.00% | 0.00% | 2.02% | 0.00% | 0.00% | 0.00% | 0.00% | 0.00% | 0.00% |
| | | | | | | | HexNAc7- | | | | | | | | | | | |
| | | | | | | | Fuc4- | | | | | | | | | | | |
| 0.00% | 0.25% | 0.25% | 8 | 7 | 5 | 0 | Hex8- | 0 | 1 | 0.00% | 0.00% | 1.78% | 0.00% | 0.00% | 0.00% | 0.00% | 0.00% | 0.00% |
| | | | | | | | HexNAc7- | | | | | | | | | | | |
| | | | | | | | Fuc5- | | | | | | | | | | | |
| 0.82% | 0.00% | 0.82% | 7 | 6 | 0 | 4 | Hex7- | 2 | 0 | 1.44% | 0.20% | 0.00% | 0.00% | 0.00% | 0.00% | 0.00% | 0.00% | 0.00% |
| | | | | | | | HexNAc6- | | | | | | | | | | | |
| | | | | | | | NeuAc4- | | | | | | | | | | | |
| 0.00% | 0.00% | 0.00% | 6 | 5 | 5 | 3 | Hex6- | 0 | 0 | 0.00% | 0.00% | 0.00% | 0.00% | 0.00% | 0.00% | 0.00% | 0.00% | 0.00% |
| | | | | | | | HexNAc5- | | | | | | | | | | | |
| | | | | | | | Fuc5- | | | | | | | | | | | |
| | | | | | | | NeuAc3- | | | | | | | | | | | |
| 0.45% | 0.00% | 0.45% | 7 | 6 | 1 | 4 | Hex7- | 1 | 0 | 0.90% | 0.00% | 0.00% | 0.00% | 0.00% | 0.00% | 0.00% | 0.00% | 0.00% |
| | | | | | | | HexNAc6- | | | | | | | | | | | |
| | | | | | | | Fuc1- | | | | | | | | | | | |
| | | | | | | | NeuAc4- | | | | | | | | | | | |
| 0.00% | 0.00% | 0.00% | 7 | 7 | 6 | 1 | Hex7- | 0 | 0 | 0.00% | 0.00% | 0.00% | 0.00% | 0.00% | 0.00% | 0.00% | 0.00% | 0.00% |
| | | | | | | | HexNAc7- | | | | | | | | | | | |
| | | | | | | | Fuc6- | | | | | | | | | | | |
| | | | | | | | NeuAc1- | | | | | | | | | | | |
| 0.00% | 0.00% | 0.00% | 7 | 7 | 7 | 1 | Hex7- | 0 | 0 | 0.00% | 0.00% | 0.00% | 0.00% | 0.00% | 0.00% | 0.00% | 0.00% | 0.00% |
| | | | | | | | HexNAc7- | | | | | | | | | | | |
| | | | | | | | Fuc7- | | | | | | | | | | | |
| | | | | | | | NeuAc1- | | | | | | | | | | | |
| 0.00% | 0.00% | 0.00% | 7 | 7 | 5 | 2 | Hex7- | 0 | 0 | 0.00% | 0.00% | 0.00% | 0.00% | 0.00% | 0.00% | 0.00% | 0.00% | 0.00% |
| | | | | | | | HexNAc7- | | | | | | | | | | | |
| | | | | | | | Fuc5- | | | | | | | | | | | |
| | | | | | | | NeuAc2- | | | | | | | | | | | |

### REFERENCES

1. Matthaei H, Schulick R D, Hruban R H, Maitra A. Cystic precursors to invasive pancreatic cancer. Nat Rev Gastroenterol Hepatol. March; 8(3):141-50.
2. Lee K S, Sekhar A, Rofsky N M, Pedrosa I. Prevalence of incidental pancreatic cysts in the adult population on MR imaging. Am J. Gastroenterol. September; 105(9):2079-84.
3. Haab B B, Porter A, Yue T, Li L, Scheiman J, Anderson M A, et al. Glycosylation variants of mucins and CEACAMs as candidate biomarkers for the diagnosis of pancreatic cystic neoplasms. Ann Surg. May; 251(5):937-45.
4. Tanaka M, Fernandez-Del Castillo C, Adsay V, Chari S, Falconi M, Jang J Y, et al. International consensus guidelines 2012 for the management of IPMN and MCN of the pancreas. Pancreatology. 2012 May; 12(3):183-97.
5. van der Waaij L A, van Dullemen H M, Porte R J. Cyst fluid analysis in the differential diagnosis of pancreatic cystic lesions: a pooled analysis. Gastrointestinal endoscopy. 2005 September; 62(3):383-9.
6. Brugge W R, Lewandrowski K, Lee-Lewandrowski E, Centeno B A, Szydlo T, Regan S, et al. Diagnosis of pancreatic cystic neoplasms: a report of the cooperative pancreatic cyst study. Gastroenterology. 2004 May; 126(5):1330-6.
7. Visser B C, Yeh B M, Qayyum A, Way L W, McCulloch C E, Coakley F V. Characterization of cystic pancreatic masses: relative accuracy of CT and MRI. AJR Am J Roentgenol. 2007 September; 189(3):648-56.
8. Singh M, Maitra A. Precursor lesions of pancreatic cancer: molecular pathology and clinical implications. Pancreatology. 2007; 7(1):9-19.
9. Khalid A, McGrath K M, Zahid M, Wilson M, Brody D, Swalsky P, et al. The role of pancreatic cyst fluid molecular analysis in predicting cyst pathology. Clin Gastroenterol Hepatol. 2005 October; 3(10):967-73.
10. Wu J, Matthaei H, Maitra A, Dal Molin M, Wood L D, Eshleman J R, et al. Recurrent GNAS Mutations Define an Unexpected Pathway for Pancreatic Cyst Development. Sci Transl Med. 2011 Jul. 20; 3(92):92ra66.
11. Raty S, Sand J, Alfthan H, Haglund C, Nordback I. Cyst fluid tumor-associated trypsin inhibitor may be helpful in the differentiation of cystic pancreatic lesions. J Gastrointest Surg. 2004 July-August; 8(5):569-74.
12. Ryu J K, Matthaei H, Dal Molin M, Hong S M, Canto M I, Schulick R D, et al. Elevated microRNA miR-21 Levels in Pancreatic Cyst Fluid Are Predictive of Mucinous Precursor Lesions of Ductal Adenocarcinoma. Pancreatology. 2011 Jul. 12; 11(3):343-50.
13. Maker A V, Katabi N, Qin L X, Klimstra D S, Schattner M, Brennan M F, et al. Cyst fluid interleukin-1beta (IL1beta) levels predict the risk of carcinoma in intraductal papillary mucinous neoplasms of the pancreas. Clin Cancer Res. 2011 Mar. 15; 17(6):1502-8.
14. Shami V M, Sundaram V, Stelow E B, Conaway M, Moskaluk C A, White G E, et al. The level of carcinoembryonic antigen and the presence of mucin as predictors of cystic pancreatic mucinous neoplasia. Pancreas. 2007 May; 34(4):466-9.
15. Hammel P R, Forgue-Lafitte M E, Levy P, Voitot H, Vilgrain V, Flejou J F, et al. Detection of gastric mucins (M1 antigens) in cyst fluid for the diagnosis of cystic lesions of the pancreas. International journal of cancer. 1997 Jun. 20; 74(3):286-90.
16. Garud S S, Willingham F F. Molecular analysis of cyst fluid aspiration in the diagnosis and risk assessment of cystic lesions of the pancreas. Clin Transl Sci. February; 5(1):102-7.
17. Khalid A, Zahid M, Finkelstein S D, LeBlanc J K, Kaushik N, Ahmad N, et al. Pancreatic cyst fluid DNA analysis in evaluating pancreatic cysts: a report of the PANDA study. Gastrointestinal endoscopy.
2009 May; 69(6):1095-102.
18. Mann B F, Goetz J A, House M G, Schmidt C M, Novotny M V. Glycomic and proteomic profiling of pancreatic cyst fluids identifies hyperfucosylated lactosamines on the N-linked glycans of overexpressed glycoproteins. Mol Cell Proteomics. July; 11(7):M111 015792.
19. Cuoghi A, Farina A, Z'Graggen K, Dumonceau J M, Tomasi A, Hochstrasser D F, et al. Role of proteomics to differentiate between benign and potentially malignant pancreatic cysts. J Proteome Res. May 6; 10(5):2664-70.
20. Haab B B, Porter A, Yue T, Li L, Scheiman J, Anderson M A, et al. Glycosylation Variants of Mucins and CEACAMs as Candidate Biomarkers for the Diagnosis of Pancreatic Cystic Neoplasms. Annals of surgery. [Research]. 2010 May 2010; 251(5):937-45.
21. Lau K S, Dennis J W. N-Glycans in cancer progression. Glycobiology. 2008 October; 18(10):750-60.
22. Wang Y, Ju T, Ding X, Xia B, Wang W, Xia L, et al. Cosmc is an essential chaperone for correct protein O-glycosylation. Proceedings of the National Academy of Sciences of the United States of America. 2010 May 18; 107(20):9228-33.
23. Lin Z, Simeone D M, Anderson M A, Brand R E, Xie X, Shedden K A, et al. Mass spectrometric assay for analysis of haptoglobin fucosylation in pancreatic cancer. J Proteome Res. May 6; 10(5):2602-11.
24. Okuyama N, Ide Y, Nakano M, Nakagawa T, Yamanaka K, Moriwaki K, et al. Fucosylated haptoglobin is a novel marker for pancreatic cancer: A detailed analysis of the oligosaccharide structure and a possible mechanism for fucosylation. International journal of cancer. 2006 Dec. 29; 118(11):2803-8.
25. Dennis J W, Granovsky M, Warren C E. Glycoprotein glycosylation and cancer progression. Biochimica et biophysica acta. 1999 Dec. 6; 1473(1):21-34.
26. Chen S, LaRoche T, Hamelinck D, Bergsma D, Brenner D, Simeone D, et al. Multiplexed analysis of glycan variation on native proteins captured by antibody microarrays. Nature methods. 2007 May; 4(5):437-44.
27. Chen M, Van Ness M, Guo Y, Gregg J. Molecular pathology of pancreatic neuroendocrine tumors. J Gastrointest Oncol. September; 3(3):182-8.
28. Partyka K, McDonald M, Maupin K A, Brand R, Kwon R, Simeone D M, et al. Comparison of surgical and endoscopic sample collection for pancreatic cyst fluid biomarker identification. Journal of proteome research. [Research Support, N.I.H., Extramural
Research Support, Non-U.S. Gov't]. 2012 May 4; 11(5):2904-11.
29. Maker A V, Katabi N, Gonen M, Dematteo R P, D'Angelica M I, Fong Y, et al. Pancreatic Cyst Fluid and Serum Mucin Levels Predict Dysplasia in Intraductal Papillary Mucinous Neoplasms of the Pancreas. Ann Surg Oncol. 2010 Aug. 18.
30. Cizginer S, Turner B, Bilge A R, Karaca C, Pitman M B, Brugge W R. Cyst fluid carcinoembryonic antigen is an accurate diagnostic marker of pancreatic mucinous cysts. Pancreas. October; 40(7):1024-8.
31. Aljebreen A M, Romagnuolo J, Perini R, Sutherland F. Utility of endoscopic ultrasound, cytology and fluid carcinoembryonic antigen and CA 19-9 levels in pancreatic cystic lesions. World J. Gastroenterol. 2007 Aug. 7; 13(29):3962-6.
32. Porter A, Yue T, Heering a L, Day S, Suh E, Haab B B. A motif-based analysis of glycan array data to determine the specificities of glycan-binding proteins. Glycobiology. 2010 March 2010; 20(3):369-80.
33. West M B, Segu Z M, Feasley C L, Kang P, Klouckova I, Li C, et al. Analysis of site-specific glycosylation of renal and hepatic gamma-glutamyl transpeptidase from normal human tissue. The Journal of biological chemistry. 2010 Sep. 17; 285(38):29511-24.
34. Babu P, North S J, Jang-Lee J, Chalabi S, Mackerness K, Stowell S R, et al. Structural characterisation of neutrophil glycans by ultra-sensitive mass spectrometric glycomics methodology. Glycoconjugate journal. 2009 November; 26(8):975-86.
35. Knezevic A, Polasek O, Gornik O, Rudan I, Campbell H, Hayward C, et al. Variability, heritability and environmental determinants of human plasma N-glycome. Journal of proteome research. 2009 February; 8(2):694-701.
36. Chen R, Jiang X, Sun D, Han G, Wang F, Ye M, et al. Glycoproteomics analysis of human liver tissue by combination of multiple enzyme digestion and hydrazide chemistry. Journal of proteome research. 2009 February; 8(2):651-61.
37. Haab B B. Antibody-lectin sandwich arrays for biomarker and glycobiology studies. Expert review of proteomics. 2010 February; 7(1):9-11.
38. Wu J T. Serum alpha-fetoprotein and its lectin reactivity in liver diseases: a review. Ann Clin Lab Sci. 1990 March-April; 20(2):98-105.
39. Miyoshi E, Nakano M. Fucosylated haptoglobin is a novel marker for pancreatic cancer: detailed analyses of oligosaccharide structures. Proteomics. 2008 August; 8(16):3257-62.
40. Parekh R B, Dwek R A, Sutton B J, Fernandes D L, Leung A, Stanworth D, et al. Association of rheumatoid arthritis and primary osteoarthritis with changes in the glycosylation pattern of total serum IgG. Nature. 1985 Aug. 1-7; 316(6027):452-7.
41. Irimura T, Denda K, Iida S, Takeuchi H, Kato K. Diverse glycosylation of MUC1 and MUC2: potential significance in tumor immunity. Journal of biochemistry. 1999 December; 126(6):975-85.
42. Chang J W, Kang U B, Kim D H, Yi J K, Lee J W, Noh D Y, et al. Identification of circulating endorepellin LG3 fragment: Potential use as a serological biomarker for breast cancer. Proteomics Clin Appl.
2008 January; 2(1):23-32.
43. Whitelock J M, Melrose J, Iozzo R V. Diverse cell signaling events modulated by perlecan. Biochemistry. 2008 Oct. 28; 47(43):11174-83.
44. Mongiat M, Sweeney S M, San Antonio J D, Fu J, Iozzo R V. Endorepellin, a novel inhibitor of angiogenesis derived from the C terminus of perlecan. The Journal of biological chemistry. 2003 Feb. 7; 278(6):4238-49.
45. Tachezy M, Reichelt U, Melenberg T, Gebauer F, Izbicki J R, Kaifi J T. Angiogenesis index CD105 (endoglin)/CD31 (PECAM-1) as a predictive factor for invasion and proliferation in intraductal papillary mucinous neoplasm (IPMN) of the pancreas. Histology and histopathology. 2010 October; 25(10):1239-46.
46. Kato S, Hokari R, Crawley S, Gum J, Ahn D H, Kim J W, et al. MUC5AC mucin gene regulation in pancreatic cancer cells. Int J Oncol. 2006 July; 29(1):33-40.
47. Torres M P, Chakraborty S, Souchek J, Batra S K. Mucin-based targeted pancreatic cancer therapy. Curr Pharm Des. 18(17):2472-81.
48. Hoshi H, Sawada T, Uchida M, Saito H, Iijima H, Toda-Agetsuma M, et al. Tumor-associated MUC5AC stimulates in vivo tumorigenicity of human pancreatic cancer. Int J Oncol. March; 38(3):619-27.
49. Hasnain S Z, Evans C M, Roy M, Gallagher A L, Kindrachuk K N, Barron L, et al. Muc5ac: a critical component mediating the rejection of enteric nematodes. J Exp Med. May 9; 208(5):893-900.
50. Inaguma S, Kasai K, Ikeda H. GLI1 facilitates the migration and invasion of pancreatic cancer cells through MUC5AC-mediated attenuation of E-cadherin. Oncogene. [Research Support, Non-U.S. Gov't]. 2011 Feb. 10; 30(6):714-23.
51. Rausch P, Rehman A, Kunzel S, Hasler R, Ott S J, Schreiber S, et al. Colonic mucosa-associated microbiota is influenced by an interaction of Crohn disease and FUT2 (Secretor) genotype. Proceedings of the National Academy of Sciences of the United States of America. 2011 Nov. 22; 108(47):19030-5.
52. Hasehira K, Tateno H, Onuma Y, Ito Y, Asashima M, Hirabayashi J. Structural and quantitative evidence for dynamic glycome shift on production of induced pluripotent stem cells. Molecular & cellular proteomics: MCP. 2012 December; 11(12):1913-23.
53. Satomaa T, Heiskanen A, Mikkola M, Olsson C, Blomqvist M, Tiittanen M, et al. The N-glycome of human embryonic stem cells. BMC cell biology. [Research Support, Non-U.S. Gov't]. 2009; 10:42.
54. Satomaa T, Heiskanen A, Leonardsson I, Angstrom J, Olonen A, Blomqvist M, et al. Analysis of the human cancer glycome identifies a novel group of tumor-associated N-acetylglucosamine glycan antigens. Cancer research. 2009 Jul. 15; 69(14):5811-9.
55. Kobayashi M, Lee H, Nakayama J, Fukuda M. Roles of gastric mucin-type O-glycans in the pathogenesis of Helicobacter pylori infection. Glycobiology. 2009 May; 19(5):453-61.
56. Basturk O, Coban I, Adsay N V. Pancreatic cysts: pathologic classification, differential diagnosis, and clinical implications. Arch Pathol Lab Med. 2009 March; 133(3):423-38.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the claims.

What is claimed is:

1. A method for diagnosing whether a pancreatic cystic lesion in a subject is malignant, the method comprising:
a. obtaining a pancreatic cyst fluid sample from a pancreatic cystic lesion in the subject;
b. detecting levels of glycoforms of MUC5AC and endorepellin in the sample;
c. comparing the glycoform levels of MUC5AC and endorepellin in the sample to control levels of these glycoforms of MUC5AC and endorepellin in control pancreatic cyst samples; and
d. diagnosing the pancreatic cystic lesion as malignant if the glycoform levels of the MUC5AC and endorepellin in the sample are differentially expressed as compared to the control levels of these glycoforms of MUC5AC and endorepellin.

2. The method of claim 1, wherein the MUC5AC glycoforms are detected using wheat-germ agglutinin (MUC5AC-WGA) and an antibody to blood group H (MUC5AC-BGH) antigen, and the endorepellin glycoform is detected using WGA (endorepellin-WGA).

3. The method of claim 2, wherein if the levels of the glycoforms detected by MUC5AC-WGA, MUC5AC-BGH, and endorepellin-WGA in the sample are elevated as compared to the control levels of these glycoforms of MUC5AC and endorepellin, the pancreatic cystic lesion is diagnosed as malignant.

4. The method of claim 2, wherein if the level of none or one of the glycoforms MUC5AC-WGA, MUC5AC-BGH, and endorepellin-WGA in the sample is elevated as compared to the control levels of these glycoforms of MUC5AC and endorepellin, the pancreatic cystic lesion is diagnosed as benign.

5. The method of claim 1, wherein the pancreatic cyst sample is obtained using endoscopic ultrasound guided fine-needle aspiration.

6. The method according to claim 1, wherein the glycoforms detected using MUC5AC-WGA, MUC5AC-BGH and endorepellin-WGA are quantified using an antibody-lectin sandwich assay.

7. The method of claim 1, wherein the malignant pancreatic cysts are mucinous cystic neoplasms or intraductal papillary mucinous neoplasms.

8. The method of claim 1, further comprising treating the subject diagnosed as having a malignant pancreatic cystic lesion.

9. The method of claim 8, wherein treatment comprises surgically resecting the malignant pancreatic cystic lesion, applying radiation to the malignant pancreatic cystic lesion or administering a regimen of chemotherapeutic agents to reduce the growth or survivability of the malignant pancreatic cystic lesion.

10. A method for determining the malignant potential of a pancreatic cyst lesion in a subject having or suspected of having pancreatic cancer, comprising:
a. obtaining a pancreatic cyst fluid sample from a pancreatic cyst lesion in the subject;
b. measuring in the sample levels of MUC5AC glycoforms detected by wheat-germ agglutinin (MUC5AC-WGA) and by an antibody to blood group H (MUC5AC-BGH);
c. measuring in the sample the level of an endorepellin glycoform using wheat-germ agglutinin (endorepellin-WGA);
d. comparing the levels of MUC5AC-WGA, MUC5AC-BGH, and endorepellin-WGA glycoforms in the sample to a statistical threshold level for MUC5AC-WGA, MUC5AC-BGH, and endorepellin-WGA glycoforms obtained from a comparable control of non-malignant pancreatic cyst lesions; and
e. determining that the pancreatic cyst lesion of the subject is a malignant pancreatic cyst if two of the three levels of MUC5AC-WGA, MUC5AC-BGH, and endorepellin-WGA glycoforms are higher in the pancreatic cyst fluid sample than the two of the three levels of MUC5AC-WGA, MUC5AC-BGH and endorepellin-WGA glycoforms in the comparable control of non-malignant pancreatic cyst lesions.

11. The method of claim 10, wherein the pancreatic cyst fluid sample is obtained using endoscopic ultrasound guided fine-needle aspiration.

12. The method of claim 10, wherein the glycoforms MUC5AC-WGA, MUC5AC-BGH, and endorepellin-WGA are quantified using an antibody-lectin sandwich assay.

13. The method of claim 12, wherein the assay is a microarray performed in high-throughput mode.

14. The method of claim 10, wherein the malignant pancreatic cyst is a mucinous cystic neoplasm or an intraductal papillary mucinous neoplasm.

15. The method of claim 10, wherein the comparable control of non-malignant pancreatic cyst lesions is a sex and/or age matched non-malignant pancreatic cyst lesion.

16. A kit comprising:
a. a substrate for depositing a discrete sample specimen;
b. at least one binding reagent, the at least one binding reagent operable to bind specifically to at least two glycoforms selected from MUC5AC glycoforms and an endorepellin glycoform present in the discrete sample specimen; and
c. a detection reagent operable to identify a complex formed between the at least one binding reagent and the at least two glycoforms.

17. The kit of claim 16, wherein the binding reagent is selected from the group consisting of wheat-germ agglutinin and an anti-blood group H antigen antibody.

18. The kit of claim 17, wherein the detection reagent is selected from a wheat-germ agglutinin antibody and a labeled secondary antibody to the anti-blood group H antigen antibody.

19. The kit of claim 16, further comprising one or more containers for the binding and detection reagents.

20. The kit of claim 16, wherein the substrate comprises: an antibody microarray having an anti-MUC5AC capture antibody and an anti-endorepellin capture antibody bound thereto.


SEQUENCE LISTING (2 sequences)

SEQ ID NO: 1 - protein, 876 aa, Homo sapiens
MSLQFNGFVRCDHTVLPTELNGLLGFPWTVGQAPGAHCGGSEMQGSEVLTCAPMPWLVWAPGNHSEGSTLWLVLSPRPTRMGIHGAHGLAGPPIREGQHVHSGAGGQGTSRQHPLTLMGLWGYQEHGLSEARPGAVLDPTAHPGLGEVALGHPRLFPQLCGAEAIHRGLSPPDVCPPVCIHLSDPEERGALAAAQDQSCGASRTRRLLRDRALFPAVHTMSVGRRKLALLWALALALACTRHTGHAQDGSSESSYKHHPALSPIARGPSGVPLRGATVFPSLRTIPVVRASNPAHNGRVCSTWGSFHYKTFDGDVFRFPGLCNYVFSEHCGAAYEDFNIQLRRSQESAAPTLSRVLMKVDGVVIQLTKGSVLVNGHPVLLPFSQSGVLIQQSSSYTKVEARLGLVLMWNHDDSLLLELDTKYANKTCGLCGDFNGMPVVSELLSHNTKLTPMEFGNLQKMDDPTEQCQDPVPEPPRNCSTGFGICEELLHGQLFSGCVALVDVGSYLEACRQDLCFCEDTDLLSCVCHTLAEYSRQCTHAGGLPQDWRGPDFCPQKCPNNMQYHECRSPCADTCSNQEHSRACEDHCVAGCFCPEGTVLDDIGQTGCVPVSKCACVYNGAAYAPGATYSTDCTNCTCSGGRWSCQEVPCPGTCSVLGGAHFSTFDGKQYTVHGDCSYVLTKPCDSSAFTVLAELRRCGLTDSETCLKSVTLSLDGVQTVVVIKASGEVFLNQIYTQLPISAANVTIFRPSTFFIIAQTSLGLQLNLQLVPTMQLFMQLAPKLRGQTCGLCGNFNSIQADDFRTLSGVVEATAAAFFNTFKTQAACPNIRNSFEDPCSLSVENGTGVHGSPLCWPPGAGSPASIWHCRQRGWPCVCL

SEQ ID NO: 2 - protein, 705 aa, Homo sapiens
EIKITFRPDSADGMLLYNGQKRVPGSPTNLANRQPDFISFGLVGGRPEFRFDAGSGMATIRHPTPLALGHFHTVTLLRSLTQGSLIVGDLAPVNGTSQGKFQGLDLNEELYLGGYPDYGAIPKAGLSSGFIGCVRELRIQGEEIVFHDLNLTAHGISHCPTCRDRPCQNGGQCHDSESSSYVCVCPAGFTGSRCEHSQALHCHPEACGPDATCVNRPDGRGYTCRCHLGRSGLRCEEGVTVTTPSLSGAGSYLALPALTNTHHELRLDVEFKPLAPDGVLLFSGGKSGPVEDFVSLAMVGGHLEFRYELGSGLAVLRSAEPLALGRWHRVSAERLNKDGSLRVNGGRPVLRSSPGKSQGLNLHTLLYLGGVEPSVPLSPATNMSAHFRGCVGEVSVNGKRLDLTYSFLGSQGIGQCYDSSPCERQPCQHGATCMPAGEYEFQCLCRDGFKGDLCEHEENPCQLREPCLHGGTCQGTRCLCLPGFSGPRCQQGSGHGIAESDWHLEGSGGNDAPGQYGAYFHDDGFLAFPGHVFSRSLPEVPETIELEVRTSTASGLLLWQGVEVGEAGQGKDFISLGLQDGHLVFRYQLGSGEARLVSEDPINDGEWHRVTALREGRRGSIQVDGEELVSGRSPGPNVAVNAKGSVYIGGAPDVATLTGGRFSSGITGCVKNLVLHSARPGAPPPQPLDLQHRAQAGANTRPCPS